(19) United States
(12) Patent Application Publication
(10) Pub. No.: US 2024/0390062 A1
(43) Pub. Date: Nov. 28, 2024

(54) CATHETER LOCKING ASSEMBLY

(71) Applicant: Medtronic Ireland Manufacturing Unlimited Company, Dublin (IE)

(72) Inventors: Jaclyn N. Kawwas, Philadelphia, PA (US); Paul J. Coates, Corte Madera, CA (US); Michael R. Neidert, San Francisco, CA (US)

(21) Appl. No.: 18/695,276

(22) PCT Filed: Sep. 16, 2022
(86) PCT No.: PCT/EP2022/075773

§ 371 Date: Mar. 25, 2024

Related U.S. Application Data
(60) Provisional application No. 63/250,715, filed on Sep. 30, 2021.

(51) Int. Cl. A61B 18/14; A61B 18/00; A61B 18/04; A61M 25/00

(52) U.S. Cl. CPC A61B 18/1492; A61B 18/04; A61B 2018/00434; A61B 2018/00511; A61B 2018/00577; A61B 2018/046; A61M 2025/0004; A61M 25/0084

(57) ABSTRACT

A medical assembly 100 includes a support catheter 102, a therapeutic catheter 104, and a locking assembly 118 coupled to at least one of the support catheter or the therapeutic catheter. The support catheter includes an elongated body defining a guide lumen and configured to position directly within vasculature of a patient. The therapeutic catheter includes an elongated body, one or more therapeutic elements 110 at a distal portion of the elongated body, and an actuation assembly. The elongated body defines a delivery lumen and is configured to move within the guide lumen. The therapeutic elements are configured to extend from the delivery lumen into a treatment site of the patient. The actuation assembly is configured to deploy the therapeutic elements from the delivery lumen into the treatment site. The locking assembly is configured to fix the therapeutic catheter relative to the support catheter while the therapeutic elements are deployed.

### TECHNICAL FIELD

The present technology is related to catheters. In particular, various examples of the present technology are related to neuromodulation catheters.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function can be considerably stimulated in disease states characterized by heightened sympathetic tone. For example, reduced renal blood flow and glomerular filtration rate as a result of renal sympathetic efferent stimulation may be a cornerstone of the loss of renal function in cardio-renal syndrome, (i.e., renal dysfunction as a progressive complication of chronic heart failure).

Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter the renal sympathetic mediated sodium and water retention). These pharmacologic strategies, however, can have significant limitations including limited efficacy, compliance issues, side effects, and others.

### SUMMARY

The present technology is directed to devices, systems, and methods for catheter stabilization during neuromodulation, such as renal neuromodulation.

In some examples, the disclosure describes a medical assembly that includes a support catheter, a therapeutic catheter, and a locking assembly coupled to at least one of the support catheter or the therapeutic catheter. The support catheter includes an elongated body defining a guide lumen and configured to position directly within vasculature of a patient. The therapeutic catheter includes an elongated body, one or more therapeutic elements, and an actuation assembly. The elongated body defines a delivery lumen and is configured to move within the guide lumen. The one or more therapeutic elements are positioned at a distal portion of the elongated body. The one or more therapeutic elements are configured to extend from the delivery lumen into a deployed configuration at a treatment site of the patient. The actuation assembly is configured to deploy the one or more therapeutic elements to extend from the delivery lumen into the deployed configuration. The locking assembly is configured to fix the therapeutic catheter relative to the support catheter while the one or more therapeutic elements are extended from the delivery lumen.

In some examples, the disclosure describes a method that includes positioning a distal portion of an elongated body of a support catheter in vasculature of a patient. The elongated body of the support catheter defines a guide lumen. The method further includes advancing a distal portion of an elongated body of a therapeutic catheter through the guide lumen to a treatment site of the patient. The elongated body defines a delivery lumen. The distal portion of the therapeutic catheter includes one or more therapeutic elements configured to extend from the delivery lumen into a deployed configuration at the treatment site. The method further includes fixing the therapeutic catheter relative to the support catheter and deploying the one or more therapeutic elements to extend from the delivery lumen into the deployed configuration.

In some examples, the disclosure describes a therapeutic catheter that includes an elongated body, one or more therapeutic elements, an actuation assembly, and a locking assembly. The elongated body defines a delivery lumen and is configured to move within a guide lumen of a support catheter. The one or more therapeutic elements are positioned at a distal portion of the elongated body. The one or more therapeutic elements are configured to extend from the delivery lumen into a deployed configuration at a treatment site of the patient. The actuation assembly is configured to deploy the one or more therapeutic elements to extend from the delivery lumen into the deployed configuration. The locking assembly is configured to fix the therapeutic catheter relative to the support catheter while the one or more therapeutic elements are extended from the delivery lumen.

In some examples, the disclosure describes a medical assembly that includes a support catheter, a therapeutic catheter, and a locking assembly coupled to at least one of the support catheter or the therapeutic catheter. The support catheter includes an elongated body defining a guide lumen and configured to position directly within vasculature of a patient. The therapeutic catheter includes an elongated body, one or more therapeutic elements, and an actuation assembly. The elongated body is configured to move within the guide lumen. The one or more therapeutic elements are positioned at a distal portion of the elongated body. The one or more therapeutic elements are configured to deliver therapy at a treatment site of a patient. The actuation assembly is configured to control delivery of the therapy from the one or more therapeutic elements. The locking assembly is configured to fix the therapeutic catheter relative to the support catheter while the one or more therapeutic elements are delivering therapy.

In some examples, the disclosure describes a method that includes positioning a distal portion of an elongated body of a support catheter in vasculature of a patient. The elongated body of the support catheter defines a guide lumen. The method further includes advancing a distal portion of an elongated body of a therapeutic catheter through the guide lumen to a treatment site of the patient. The distal portion of the therapeutic catheter includes one or more therapeutic elements configured to deliver therapy at the treatment site. The method further includes fixing the therapeutic catheter relative to the support catheter and delivering the therapy from the one or more therapeutic elements.

In some examples, the disclosure describes a therapeutic catheter that includes an elongated body, one or more therapeutic elements, an actuation assembly, and a locking assembly. The elongated body is configured to move within a guide lumen of a support catheter. The one or more therapeutic elements are positioned at a distal portion of the elongated body. The one or more therapeutic elements are configured to deliver therapy at a treatment site of a patient. The actuation assembly is configured to control delivery of the therapy from the one or more therapeutic elements. The locking assembly is configured to fix the therapeutic catheter relative to the support catheter while the one or more therapeutic elements are delivering therapy.

In some examples, the disclosure describes a locking assembly that includes a housing and a moveable element. The housing is configured to couple to at least one of a support catheter or a therapeutic catheter. The moveable element is configured to move relative to the housing between a locked position and an unlocked position and, in the locked position, fix the therapeutic catheter relative to the support catheter while one or more therapeutic elements of the therapeutic catheter are at least one of: extending from a delivery lumen of the therapeutic catheter, or delivering therapy to a tissue.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same reference numeral designations represent similar elements throughout and wherein:

FIG. 1 is a conceptual diagram illustrating an example medical assembly configured in accordance with some examples of the present disclosure.

FIG. 2A illustrates accessing of a renal artery and modulating renal nerves with the assembly of FIG. 1 in accordance with some examples of the present disclosure.

FIG. 2B illustrates a distal portion of the medical assembly of FIG. 1 in a vessel shown in cross-section, in accordance with some examples of the present disclosure.

FIG. 2C illustrates a distal portion of another embodiment of the medical assembly of FIG. 1 in a vessel shown in cross-section, in accordance with some examples of the present disclosure.

FIG. 2D illustrates a distal portion of another embodiment of the medical assembly of FIG. 1 in a vessel shown in cross-section, in accordance with some examples of the present disclosure.

FIGS. 3A-3D are side-view cross-sectional conceptual diagrams of an example medical assembly in accordance with some examples of the present disclosure.

FIGS. 4A-4D are side-view cross-sectional conceptual diagrams of an example medical assembly in accordance with some examples of the present disclosure.

FIGS. 5A-5D are side-view cross-sectional conceptual diagrams of an example medical assembly in accordance with some examples of the present disclosure.

FIGS. 6A-6D are side-view cross-sectional conceptual diagrams of an example medical assembly in accordance with some examples of the present disclosure.

FIGS. 7A-7D are side-view cross-sectional conceptual diagrams of an example medical assembly in accordance with some examples of the present disclosure.

FIGS. 8A-8D are side-view cross-sectional conceptual diagrams of an example medical assembly in accordance with some examples of the present disclosure.

FIGS. 9A-9D are side-view cross-sectional conceptual diagrams of an example medical assembly in accordance with some examples of the present disclosure.

FIG. 10 is a flow diagram illustrating an example technique for positioning and deploying therapeutic elements for neuromodulation of nerves with an interlock between securing a medical assembly and deploying the therapeutic elements, in accordance with some examples of this disclosure.

FIG. 11 is a flow diagram illustrating an example technique for positioning and deploying therapeutic elements for neuromodulation of nerves with a forced order of operations of securing a medical assembly and subsequently deploying the therapeutic elements, in accordance with some examples of this disclosure.

### DETAILED DESCRIPTION

The present technology is directed to devices, systems, and methods for catheter safety and stabilization during neuromodulation, such as renal neuromodulation.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

Various medical procedures, such as ablation procedures and expansion procedures, may involve accessing tissues of or near vasculature of a patient. As one example, vasculature expansion procedures may involve positioning and expanding a device in vasculature of a patient, such as neurovasculature, coronary vasculature, and peripheral vasculature. As another example, ablation procedures may involve accessing and ablating tissues, such as nerves, near vasculature of a patient, such as coronary vasculature for cardiac ablation or renal vasculature for renal neuromodulation.

For example, renal neuromodulation may be used to treat a variety of conditions, such as hypertension, heart failure, and chronic kidney disease, by modulating activation of the renal sympathetic neural system. Renal sympathetic nerves of the renal sympathetic nervous system generally are near or within walls of renal arteries, such that the renal arteries may provide access to the renal sympathetic nerves. For example, renal neuromodulation treatment may involve positioning one or more therapeutic elements within a lumen of a renal artery, in a wall of the renal artery, or in tissue surrounding the renal artery to access the renal sympathetic nerves. Renal neuromodulation treatments, such as renal denervation, may be accomplished using one or more of a variety of treatment modalities, including radio frequency (RF) energy, microwave energy, ultrasound energy, a chemical agent, or the like.

As one example, chemical ablation may operate by injecting a chemical agent into tissues near the renal artery to chemically ablate the renal sympathetic nerves. The chemical agent may be selected to modulate activity of one or more renal nerves adjacent to the renal artery in which the neuromodulation catheter is positioned. For example, the chemical agent may be a neurotoxic chemical selected to chemically ablate the one or more renal nerves near the renal artery. In some instances, this injection may be performed by first inserting needles from an intravascular medical device through a wall of the renal artery and then injecting a chemical agent through the needles into tissues near the renal artery to chemically ablate the renal sympathetic nerves. In other instances, this injection may be performed by delivering the chemical agent under pressure without penetrating the renal artery with a needle or other delivery device. As another example, radiofrequency (RF) ablation or stimulation may operate by positioning electrodes within a lumen or through a wall of the renal artery and delivering RF energy into tissues within or near the renal artery to thermally ablate or electrically stimulate the renal sympathetic nerves. Parameters of the RF energy, such as amplitude and frequency, may be selected to modulate activity of one or more renal nerves adjacent to the renal artery in which the neuromodulation catheter is positioned.

During a therapy procedure, such as ablation or stimulation, a clinician may anesthetize a patient and guide a support catheter through the vasculature of the patient near a treatment site of the patient's renal artery. After, or concurrent with, placement of the support catheter, the clinician may advance a therapeutic catheter through the support catheter to a treatment site. The therapeutic catheter may include one or more therapeutic elements, such as needles, electrodes, apertures, or other structures, from which a mode of therapy, such as a chemical agent or RF energy, is delivered. In some examples, the therapeutic elements may be extendable from the therapeutic catheter, such that the clinician may operate an actuation assembly of the therapeutic catheter to insert the therapeutic elements into, near, or through the wall of the renal artery and, separately, apply the therapy into the tissues near the renal artery via the therapeutic elements. In other examples, the therapeutic elements may be fixed to the therapeutic catheter, such that the clinician may operate the actuation assembly to apply the therapy into the tissue near the renal artery via the therapeutic elements. After applying the therapy, the clinician may withdraw the therapeutic elements back into the therapeutic catheter and, after withdrawing the therapeutic elements, remove the therapeutic catheter from the treatment site, such as complete removal from the support catheter or partial removal and introduction to a different treatment site. Once the therapeutic catheter is withdrawn from the patient, the clinician may remove the support catheter from the patient to complete the procedure.

In some instances, while the therapeutic catheter is still within the vessel of the patient, the therapeutic catheter may inadvertently move prior to the therapeutic elements completing delivery of therapy and/or being withdrawn into the therapeutic catheter. For example, the therapeutic elements may be only partially withdrawn from the wall of the renal artery, or the clinician may forget to cease operation of the therapeutic elements or withdraw the therapeutic elements prior to moving or removing the therapeutic catheter. As another example, the patient may clench or otherwise move, such that the support catheter may remain stable within the patient, but the therapeutic catheter may move relative to the treatment site.

In accordance with techniques of this disclosure, a medical assembly is configured to secure the therapeutic catheter to the support catheter while therapeutic elements are exposed outside the therapeutic catheter to reduce movement of therapeutic catheter during delivery of therapy to tissues or vessels of the patient, such as while the therapeutic elements are exposed to the tissues or vessels or while the therapeutic elements are delivering energy to the tissues or vessels. The medical assembly includes a support catheter that is positioned directly within vasculature of a patient and a therapeutic catheter that moves within a guide lumen of the support catheter. In response to operation of an actuation assembly, one or more therapeutic elements at a distal portion of the therapeutic catheter may extend from a delivery lumen of the therapeutic catheter into a deployed configuration and/or deliver energy or another therapy to tissues near the vasculature. A locking assembly is coupled to (or part of) the support catheter and/or the therapeutic catheter and secures the therapeutic catheter relative to the support catheter while the one or more therapeutic elements are extended from the delivery lumen or delivering energy to the tissues. For example, a locking assembly coupled to the support catheter may exert a radially inward force on an outer surface of the therapeutic catheter to fix the therapeutic catheter to the support catheter; alternatively, a locking assembly coupled to the therapeutic catheter may exert a radially outward force on an inner surface of the support catheter to fix the support catheter to the therapeutic catheter, or the like.

The locking assembly may interface with the actuation assembly of the therapeutic catheter. In some examples, the locking assembly may be integrated with an actuation element, such as a button, knob, or slidable sheath, that both deploys the therapeutic elements and fixes the therapeutic catheter to the support catheter. For example, the locking assembly may translate an actuation force used to deploy the therapeutic elements into a locking force that fixes the therapeutic catheter relative to the support catheter as long as the therapeutic elements are deployed. In some examples, the locking assembly may force an order of operations for an actuation element, such as by exposing the actuation element only after the therapeutic catheter is fixed to the support catheter. For example, the locking assembly may translate an access force used to expose the actuation element to a locking force that fixes the therapeutic catheter to the support catheter prior to the actuation element being operated. Once the therapeutic elements are partially or fully withdrawn into the therapeutic catheter or have stopped delivering energy to the tissues, the locking mechanism may unsecure the therapeutic catheter relative to the support catheter to enable movement of the therapeutic catheter in the support catheter. In any of these various ways, medical assemblies described herein may reduce or prevent movement of the therapeutic catheter relative to the support catheter while the therapeutic catheter is providing therapy to the tissues.

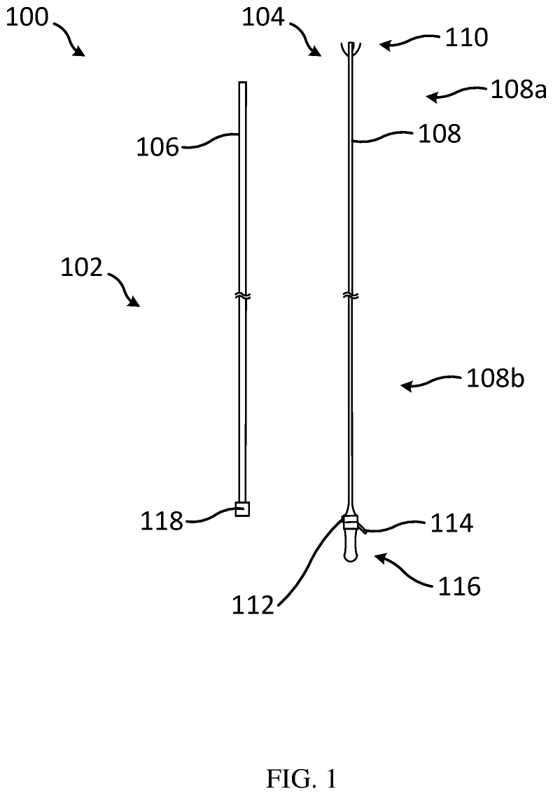

FIG. 1 is a conceptual diagram illustrating an example medical assembly 100 (“assembly 100”) configured in accordance with some examples of the present technology. As shown in FIG. 1, assembly 100 includes a support catheter 102, a therapeutic catheter 104, and a locking assembly 118. Support catheter 102 may be configured to provide support and guidance to therapeutic catheter 104 while therapeutic catheter 104 is advanced to a treatment site. Support catheter 102 includes an elongated shaft 106 defining an inner lumen (“guide lumen”). A distal portion of elongated shaft 106 is configured to be positioned directly within vasculature of a patient. The guide lumen is configured to receive at least a distal portion of an elongated shaft 108 of therapeutic catheter 104.

Therapeutic catheter 104 may be configured to provide therapy to a treatment site accessible through vasculature (or other anatomical lumen) of a patient, such as a treatment site within or beyond the vasculature. Therapeutic catheter 104 includes a handle 116 and elongated shaft 108 attached to handle 116. Handle 116 may function as a hub for operating therapeutic catheter 104. In the example of FIG. 1, handle 116 includes an actuation element 112 configured to operate an actuation assembly of therapeutic catheter 104 (not shown) and a therapy port 114 configured to receive a therapy fluid or energy, such as a chemical agent and/or other fluid for injection through a needle, aperture, or other injection apparatus, or electricity for powering an electrode.

Elongated shafts 106 and/or 108 may have any suitable outer diameter, and the diameter can be constant along the length of elongated shafts 106 and/or 108 or may vary along the length of elongated shaft 106 and/or 108. In some examples, elongated shafts 106 and/or 108 can be 2, 3, 4, 5, 6, or 7 French or another suitable size.

Elongated shaft 108 may include a distal portion 108a and a proximal portion 108b. Distal portion 108a of therapeutic catheter 102 includes one or more therapeutic elements 110. In some examples, therapeutic elements 110 are positioned around (e.g., distributed around) a circumference of distal portion 108a. Although distal portion 108a is shown in FIG. 1 as including three therapeutic elements 110 positioned around a circumference of distal portion 108a at a single longitudinal position along elongated shaft 108, in other examples, distal portion 108a may include any number of therapeutic elements 110, such as at various circumferential or longitudinal positions. By having therapeutic elements 110 located around a circumference of distal portion 108a, therapeutic catheter 104 may be used to deliver the therapy around a circumference of the anatomical lumen in which distal portion 108a is positioned.

In the example of FIG. 1, therapeutic elements 110 are configured to deliver therapy by extending from the delivery lumen of therapeutic catheter 102 and delivering a chemical agent, energy, or other therapeutic agent to the tissues. However, in other examples, therapeutic elements 110 may be fixed to therapeutic catheter 102 and deliver the therapeutic agent from therapeutic catheter, such as segmented or ring electrodes on a surface of therapeutic catheter 102, an ultrasound generator within or on the surface of therapeutic catheter 102, or a chemical agent delivery apparatus that injects a chemical agent without penetration of the chemical agent delivery apparatus through a wall of a vessel.

Therapeutic elements 110 may be configured to remain within therapeutic catheter 102 in a delivery configuration, such as within the delivery lumen, and extend from the delivery lumen of therapeutic catheter 102 into a deployed configuration. When extended into the deployed configuration at a treatment site of the patient, therapeutic elements 110 may contact or puncture a wall of the vasculature at the treatment site. In examples in which therapeutic catheter 104 is configured to deliver a chemical agent, therapeutic elements 110 may include needles configured to be deployed to extend radially from distal portion 108a and at least partially pierce a wall of the anatomical lumen in which distal portion 108a is positioned. The needles may extend to and/or through the intima, media, and/or adventitia of the wall and be configured to deliver the chemical agent to the adventitia and/or peri-adventitia, in which renal nerves are located. In other examples, therapeutic elements 110 may include needle electrodes configured to be deployed to extend radially from distal portion 108a and at least partially pierce a wall of the anatomical lumen in which distal portion 108a is positioned, or other structures configured to contact and/or pierce a wall of the anatomical lumen in which distal portion 108a is positioned. In other examples, therapeutic elements 110 may include one or more fixed electrodes configured to remain within the anatomical lumen in which distal portion 108a is positioned and deliver therapy from within the anatomical lumen.

While only partially shown in FIG. 1, therapeutic catheter 104 includes an actuation assembly configured to deploy therapeutic elements 110 to extend from the delivery lumen of therapeutic catheter 104 into the deployed configuration. The actuation assembly may include actuation element 112 operable by a clinician to deploy or withdraw therapeutic elements 110 from the delivery lumen. The actuation assembly may include any of a variety of mechanisms extend and retract therapeutic agents from and into the delivery lumen, such as a hollow guidewire, a hollow push wire, a telescoping tube, or any other mechanism translating movement of actuation element 112 to extension or retraction of therapeutic elements 110.

Locking assembly 118 may be coupled to at least one of support catheter 102 or therapeutic catheter 104. In the example of FIG. 1, locking assembly 118 is illustrated as coupled to a proximal portion of elongated shaft 106 of support catheter 102; however, in other examples, locking assembly 118 may be coupled to therapeutic catheter 102, such as proximal portion 108b of elongated shaft 108 or as part of handle 116. Locking assembly 118 may be configured to substantially restrain the therapeutic catheter 104 relative to support catheter 102 while the one or more therapeutic elements are extended from the delivery lumen, such that therapeutic catheter 104 is fixed to support catheter 102. For example, locking assembly 118 may interface with the actuation assembly of therapeutic catheter 104 to integrally tie or condition deployment of therapeutic elements 110 with fixation of therapeutic catheter 104 to support catheter 102. Locking assembly 118 may achieve this fixation by coupling to one of support catheter 102 or therapeutic catheter 104 and applying a locking force to the other of support catheter 102 or therapeutic catheter. This locking force may resist an axial force applied to therapeutic catheter 104, such that therapeutic catheter 104 may not move relative to support catheter 102 while locking assembly 118 is applying the locking force. As one example, the locking force applied by locking assembly 118 may be greater than an axial force typically applied by a clinician to move therapeutic catheter 104 in support catheter 102, such as an inadvertent force applied by the clinician. As another example, the locking force applied by locking assembly 118 may be greater than an axial force typically experienced from incidental movement of a patient during a procedure, such as clenching movement.

Distal portion 108a of elongated shaft 108 is configured to be moved within an anatomical lumen of a human patient to locate therapeutic elements 110 at a target site within or otherwise proximate to the anatomical lumen. For example, elongated shaft 108 may be configured to position therapeutic elements 110 within a blood vessel, a ureter, a duct, an airway, or another naturally occurring anatomical lumen within the human body. In certain examples, intravascular delivery of the therapeutic elements 110 includes percutaneously inserting support catheter 102 into an anatomical lumen of a patient and moving elongated shaft 108 and/or therapeutic elements 110 through the guide lumen of support catheter 102 until therapeutic elements 110 reaches a target site (e.g., a renal artery).

Once at the target site, therapeutic elements 110 can be configured to deliver therapy, such as RF energy, microwave energy, ultrasound energy, a chemical agent, or the like, to provide or facilitate neuromodulation therapy at the target site. For ease of description, the following discussion will be primarily focused on delivering a chemical agent, such as a neurotoxic chemical, in which examples therapeutic elements 110 include needles. It will be understood, however, that therapeutic elements 110 may include elements or structures configured to deliver other types of therapy. For example, therapeutic elements 110 may include needle electrodes configured to deliver RF energy for RF ablation of nerves near the anatomical lumen in which therapeutic catheter 104 is positioned, ring or segmented electrodes configured to deliver RF energy for RF ablation, or apertures configured to deliver a chemical agent using pressure and without penetration through a wall of a vessel.

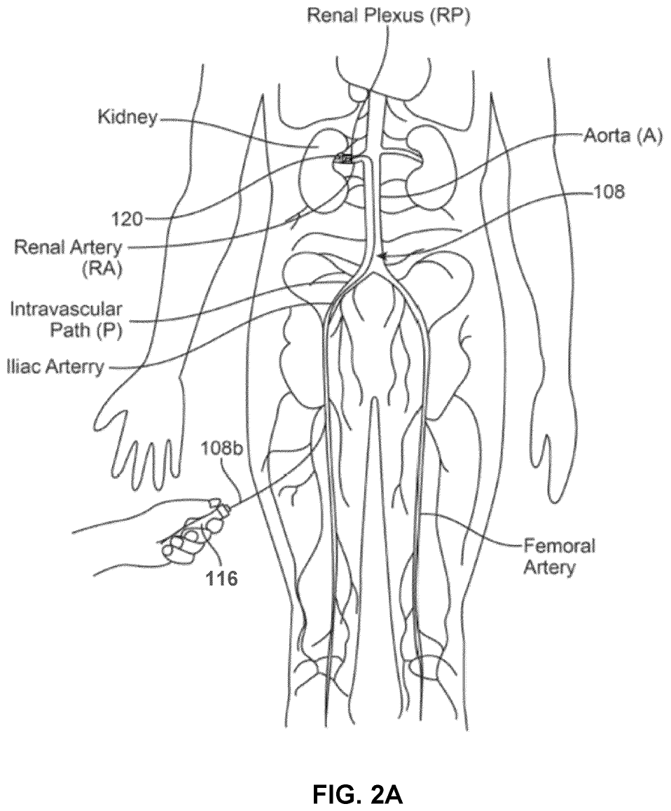

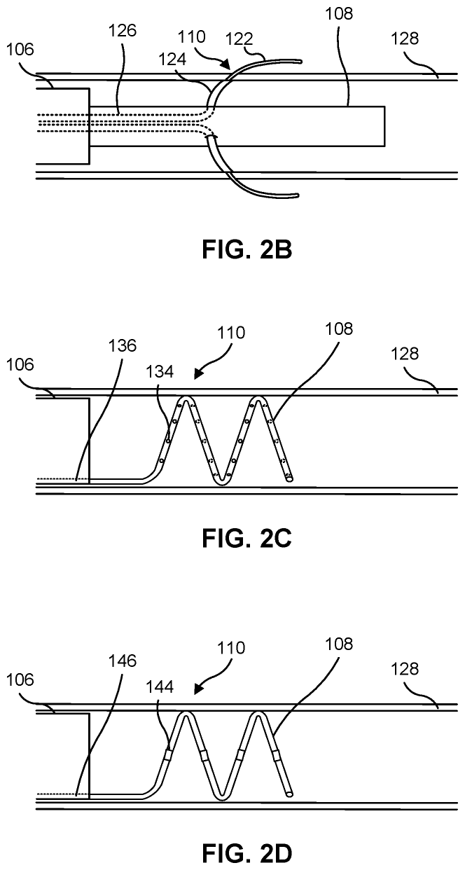

FIG. 2A (with additional reference to FIG. 1) illustrates gaining access to renal nerves of an example patient in accordance with some examples of the present technology. Therapeutic catheter 104 provides access to the renal plexus RP through an intravascular path P defined by support catheter 102 (not shown) positioned within vasculature, such as a percutaneous access site in the femoral (illustrated), brachial, radial, or axillary artery to a targeted treatment site 120 within a respective renal artery RA. In the example illustrated in FIG. 2A, therapeutic elements 110 are delivered intravascularly to treatment site 120 using support catheter 102 (not shown) to guide distal portion 108a. As noted previously, support catheter 102 may define a passageway for receiving therapeutic catheter 104 for delivery of therapeutic catheter 104 to treatment site 120. By manipulating proximal portion 108b of elongated shaft 108 from outside the intravascular path P, a clinician may advance at least distal portion 108a of elongated shaft 108 through support catheter 102 positioned in the sometimes tortuous intravascular path P and remotely manipulate distal portion 108a (FIG. 1) of elongated shaft 108.

At treatment site 120, a clinician may operate an actuation assembly to extend therapeutic elements 110 from the delivery lumen of therapeutic catheter 104 or otherwise be moved to a deployed configuration for delivering a chemical agent. While therapeutic element 110 are deployed, locking assembly 118 (not shown) may secure therapeutic catheter 104 relative to support catheter 102 while therapeutic elements 110 are extended from the delivery lumen of therapeutic catheter 104.

FIG. 2B illustrates a distal portion of medical assembly 100 of FIG. 1 in a vessel 128 shown in cross-section, in accordance with some examples of the present disclosure. A distal end of elongated shaft 108 of therapeutic catheter 104 may extend from a distal end of elongated shaft 106 of support catheter 102, such that therapeutic elements 110 may access treatment site 120 in vessel 128. Therapeutic elements 110 may each include a guide tube 124 and a needle 122 extending from guide tube 124. Needle 122 may be mechanically and fluidically coupled to a hollow extension element 126, such as a hollow tube, that delivers a chemical agent to needle 122 for injection into a tissue. As shown in FIG. 2B, needle 122 extends from guide tube 124 and therapeutic catheter 104 in a deployed configuration to pierce a wall of vessel 128.

Referring back to FIG. 2A, therapeutic elements 110 may deliver the chemical agent to treatment site 120 to cause renal neuromodulation of the renal plexus. Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

While illustrated in FIGS. 2A and 2B as using a chemical agent, renal neuromodulation can be electrically induced, thermally-induced, chemically-induced, or induced in another suitable manner or combination of manners at one or more suitable target sites during a treatment procedure. The target site can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery. Further, while described herein as therapy for renal neuromodulation, medical assemblies described herein may be used for any of a variety of therapies for various tissues or other therapeutic sites. For example, medical assemblies with needles could be used to deliver therapeutic agents locally to a tumor, deliver microwave or RF energy to a tumor, deliver therapeutic agents to modify coronary artery lesions, or the like.

FIG. 2C illustrates a distal portion of another embodiment of medical assembly 100 of FIG. 1 in a vessel 128 shown in cross-section, in accordance with some examples of the present disclosure. A distal end of elongated shaft 108 of therapeutic catheter 104 may extend from a distal end of elongated shaft 106 of support catheter 102, such that therapeutic elements 110 may access treatment site 120 in vessel 128. Therapeutic elements 110 may each include an aperture 134 configured to deliver a chemical agent to treatment site 120 without penetrating vessel 128, such as by delivering a fluid jet of the chemical agent to treatment site 120. Aperture 134 may be fluidically coupled to a hollow extension element 136, such as a hollow tube, that delivers a chemical agent under pressure from a pressurized fluid source to aperture 134 for injection into a tissue. Flow of the chemical agent may be controlled by a valve or other flow regulating device. As shown in FIG. 2C, therapeutic catheter 104 expands into a deployed configuration to position one or more apertures 134 at or near a wall of vessel 128. For example, one or more apertures 134 can be positioned at or near a wall of vessel 128 in the deployed configuration by a shape of the elongated shaft 108 (e.g., as illustrated in FIG. 2C), by a shape of one or more tubular elements, by one or more expanded balloons, and/or by other expanded part(s) of the therapeutic catheter 104.

FIG. 2D illustrates a distal portion of another embodiment of medical assembly 100 of FIG. 1 in a vessel 128 shown in cross-section, in accordance with some examples of the present disclosure. A distal end of elongated shaft 108 of therapeutic catheter 104 may extend from a distal end of elongated shaft 106 of support catheter 102, such that therapeutic elements 110 may access treatment site 120 in vessel 128. Therapeutic elements 110 may each include an electrode 144 configured to deliver energy to treatment site 120, with or without penetrating vessel 128. Electrode 144 may be electrically coupled to an electrical conductor 146 that delivers current to electrode 144 for delivery to a tissue. As shown in FIG. 2D, elongated shaft 108 expands into a deployed configuration to position one or more electrodes 144 at or near a wall of vessel 128.

During application of the therapy, the locking assembly of medical assembly 100 may secure therapeutic catheter 104 to support catheter 102, such that movement of the patient or force exerted by the clinician may not cause substantial relative movement between therapeutic elements 110 and treatment site 120. Once the therapy is applied to the treatment site, therapeutic elements 110 may be withdrawn into therapeutic catheter 104 and/or may cease operation (e.g., cease receiving electricity, pressure, or a chemical agent), and the locking assembly may unsecure therapeutic catheter 104 from support catheter 102. Distal portion 108a of therapeutic catheter 104 may be withdrawn from the treatment site into and/or through support catheter 102, such as by manipulating proximal portion 108b of elongated shaft 108.

Medical assemblies described herein may use a variety of different configurations of locking assemblies to secure a therapeutic catheter to a support catheter. Each locking assembly is coupled to (e.g., permanently or removably) at least one of therapeutic catheter 104 or support catheter 102 and configured to conditionally fix to the other of the therapeutic catheter 104 or support catheter 102 while therapeutic elements 110 are extended from the therapeutic catheter 104. This conditional fixation of the therapeutic catheter 104 to the support catheter 102 may be achieved using a variety of mechanisms.

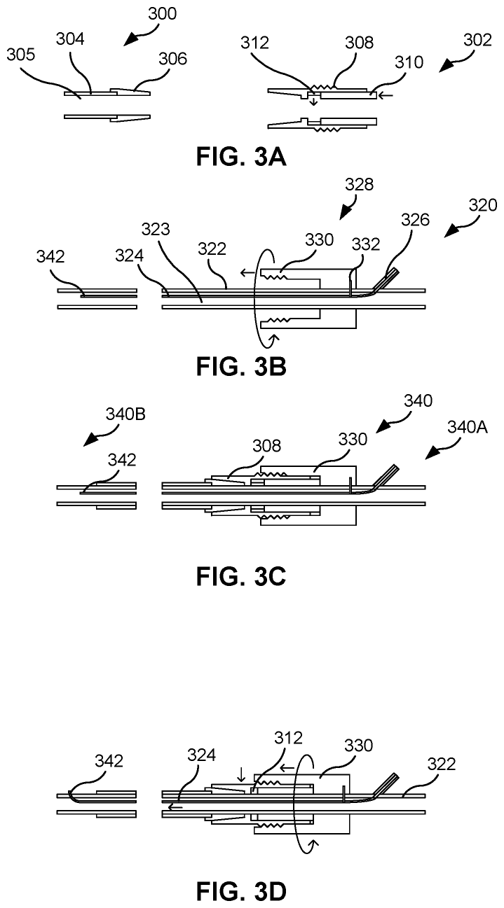

In some examples, such as will be described in FIGS. 3A-3D, 4A-4D, and 5A-5D, a locking assembly may be configured to couple to a support catheter, interface with an actuation assembly of a therapeutic catheter, and translate an actuation force for operating the actuation assembly into a locking force for fixing the therapeutic catheter to the support catheter. For example, a clinician may exert an actuation force on an actuation element of the actuation assembly to deploy one or more therapeutic elements from the therapeutic catheter. The locking assembly may interface with a portion of the actuation assembly, such as a member translating the actuation force to an extension element for deploying the therapeutic elements, to receive at least a portion of this actuation force and translate the portion of the actuation force into a locking force, such as a radially inward force, on the therapeutic catheter. By directly relating the actuation force used to deploy the therapeutic elements to the locking force used to fix the therapeutic catheter to the support catheter, locking mechanisms described herein may ensure that the therapeutic catheter is locked to the support catheter prior to or concurrent with deployment of the therapeutic elements.

In some examples, the locking mechanism is configured to couple to the support catheter and translate a rotational actuation force received by the actuation assembly of the therapeutic catheter to a radial locking force to lock the therapeutic catheter to the support catheter. FIGS. 3A-3D describe a medical assembly 340 that includes a support catheter 300, a locking assembly 302 coupled to support catheter 300, and a therapeutic catheter 320 operable by a rotatable actuation assembly 328.

FIG. 3A is a side-view cross-sectional conceptual diagram of a proximal portion of support catheter 300 and rotational locking assembly 302 of an example medical assembly 340. Support catheter 300 includes an elongated body 304, such as elongated shaft 106 of FIG. 1, defining a guide lumen 305 and a proximal connection luer 306. Locking assembly 302 is configured to interface with actuation assembly 328 of therapeutic catheter 320 and translate a rotational force from therapeutic catheter 320 to a radial force. A rotational force may include any force generated from rotation of an actuation element, including derivative forces generated from a combination of rotational movement with other structures or surfaces of locking assembly 302 or therapeutic catheter 320.

In the example of FIG. 3A, locking assembly 302 is illustrated as a fixed portion of a Tuohy Borst fitting configured to translate rotation of an external rotational cap into an inward radial force; however, in other examples, locking assembly 302 may include other assemblies configured to translate a rotational force into an inward radial force. Locking assembly 302 includes an outwardly threaded housing 308, a stiff compression sleeve 310, and a flexible elastic sleeve 312 configured to deform radially inward in response to a compressive force from compression sleeve 310. Compression sleeve 310 may be configured to resist deformation in response to an axial force, while elastic sleeve 312 may be configured to deform in response to an axial force. As illustrated in FIG. 3A, a distal axial force on compression sleeve 310 may create an inward radial force exerted by elastic sleeve 312 due to compression of elastic sleeve 312. However, in other examples, locking assembly 302 may include a collet-type mechanism configured to reduce a radius of locking assembly 302 and exert an inward radial force.

FIG. 3B is a side-view cross-sectional conceptual diagram of therapeutic catheter 320 of example medical assembly 340 having rotational actuation assembly 328. Therapeutic catheter 320 includes actuation assembly 328, an elongated body 322 defining a delivery lumen 323, and a fluid port 326. In the example of FIG. 3B, actuation assembly 328 is illustrated as a rotating portion of a Tuohy Borst fitting configured to interface with locking assembly 302 and receive a rotational force on an external rotational housing; however, in other examples, actuation assembly 328 may include other assemblies configured to receive a rotational force. Rotational actuation assembly 328 includes an inwardly threaded housing 330, an intermediate member 332, and an extension member 324.

Extension member 324 is mechanically coupled to one or more therapeutic elements 342 (illustrated as a single therapeutic element 342) at a distal end and fluidically coupled to fluid port 326 at a proximal end. Extension member 324 includes an inner lumen (“member lumen”) configured to permit passage of fluid, such as a chemical agent, to therapeutic element 342. However, in other examples, fluid port 326 may be a therapy port configured to receive a therapy, extension member 324 may be configured to deliver the therapy to therapeutic element 342, and therapeutic element 342 may be configured to deliver the therapy to a tissue. For example, fluid port 326 may be a connector configured to receive current from an energy generator, such as an RF generator, extension member 324 may include an electrical conductor configured to deliver the current from the RF generator, and therapeutic element 342 may include an electrode configured to receive the current and delivery energy to tissues.

Extension member is mechanically coupled to intermediate member 332 and configured to axially move within a delivery lumen of elongated body 322 in response to axial movement of intermediate member 332. Intermediate member 332 is configured to translate axial movement of housing 330 into axial movement of extension member 324, such that distal axial movement of housing 330 distally advances therapeutic element 342 from therapeutic catheter 320.

FIG. 3C is a side-view cross-sectional conceptual diagram of example medical assembly 340 in an undeployed configuration. Medical assembly 340 may be assembled and positioned such that a distal end 340B may be positioned at a treatment site within a vessel of a patient and a proximal end 340A may be configured to create an interface to translate an actuation force on actuation assembly 328 into a locking force exerted by locking assembly 302. At distal end 340B, therapeutic catheter 320 may extend from support catheter 300, such that therapeutic element 342 may extend from therapeutic catheter 320 to the treatment site. At proximal end 340A, inward threads from housing 330 may engage with outward threads from housing 308, such that rotation of housing 308 may distally advance housing 308 relative to elongated body 322 of therapeutic catheter 320.

FIG. 3D is a side-view cross-sectional conceptual diagram of the example medical assembly of FIG. 3C in a deployed configuration. To deploy therapeutic element 342, housing 330 may be rotated, such that threads of housing 330 may engage threads of housing 308 to distally advance housing 330. As a result, extension member 324 may distally advance therapeutic element 342 to extend from elongated body 322 of therapeutic catheter 320. The distal advancement of housing 330 may also exert an axial force on elastic sleeve 312 via compression sleeve 310 to generate a radially inward force on an outer surface of elongated body 322 of therapeutic catheter 320. This radially inward force may fix therapeutic catheter 320 to support catheter 300, such that therapeutic catheter 320 may not move axially within guide lumen 303 of support catheter 300. To withdraw therapeutic element 342, housing 330 may be rotated in an opposite direction, such that extension member 324 may proximally withdraw therapeutic element 342 into therapeutic catheter 320. Elastic sleeve 312 may stop exerting an inward radial force on elongated body 322, such that therapeutic catheter 320 may be moved relative to support catheter 300.

Locking assembly 328 may be configured to attach at multiple locations of support catheter 300. For example, to apply therapy to different treatment sites, a clinician may reposition therapeutic catheter 320 within support catheter 300. Prior to repositioning therapeutic catheter 320, locking assembly 328 may be uncoupled from a first position on support catheter 300 and reattached at a second position on support catheter 300, such as by moving connection luer 302. The second position may correspond to a particular treatment site for therapeutic catheter 320.

In this way, locking assembly 302 may translate the actuation force created through rotation from actuation assembly 328 into a radial force that secures therapeutic catheter 320 to support catheter 300. In some instances, the rotational mechanism of rotational actuation assembly 328 may enable a timing of deployment of therapeutic element 342 and fixation of therapeutic catheter 320 to support catheter 300 to be tuned, as rotation may enable various discrete or continuous degrees of actuation, and/or may permit finer control over deployment of therapeutic element 342, such as for a particular depth of penetration. For example, a timing of locking assembly 302 may be tuned such that a first position of rotational actuation assembly 328 may correspond to exerting the radially inward force, a second position of rotational actuation assembly 328 may correspond to extending therapeutic element 342 from therapeutic catheter 320, and a third position of rotational actuation assembly 328 may correspond to therapeutic element 342 contacting a wall of the vessel.

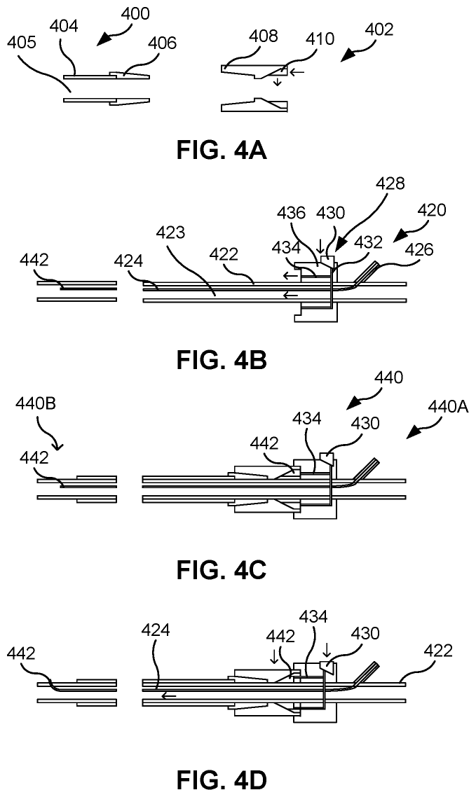

In some examples, the locking assembly is configured to couple to a support catheter and translate a radial actuation force used to operate an actuation assembly of a therapeutic catheter to a radial force to lock the therapeutic catheter to the support catheter. For example, a radial actuation force may be applied at a single time, such as through pushing a button, such that exertion of the actuation force by a clinician and exertion of the locking force by the locking assembly may occur substantially simultaneously. FIGS. 4A-4D describe a medical assembly 440 that includes a support catheter 400, a locking assembly 402 coupled to support catheter 400, and a therapeutic catheter 420 operable by a pushable actuation assembly 428.

FIG. 4A is a side-view cross-sectional conceptual diagram of a proximal portion of support catheter 400 and pushable locking assembly 402 of example medical assembly 440. Support catheter 400 includes an elongated body 404 defining a guide lumen 405 and a proximal connection luer 406, such as described with respect to elongated body 304, guide lumen 305, and connection luer 306 of FIG. 3A. Locking assembly 402 is configured to interface with pushable actuation assembly 428 of therapeutic catheter 420 and translate a radial force received by therapeutic catheter 420 to an inward radial force. A radial force may include any force generated from radial movement of an actuation element, including derivative forces generated from radial movement with other structures or surfaces of locking assembly 402 or therapeutic catheter 420.

In the example of FIG. 4A, locking assembly 402 is illustrated as a tapered wedge fitting configured to translate an inward radial force, into an axial force, and into an inward radial force; however, in other examples, locking assembly 402 may include other assemblies configured to translate a radial force received by therapeutic catheter 420 into a radial force exerted by support catheter 400. Locking assembly 402 includes a tapered housing 408 that includes a tapered surface and a tapered element 410 configured to advance radially inward in response to an axial force in a distal direction.

FIG. 4B is a side-view cross-sectional conceptual diagram of therapeutic catheter 420 of example medical assembly 440 having a pushable actuation assembly 428. Therapeutic catheter 420 includes actuation assembly 428, an elongated body 422 defining a delivery lumen 423, and a fluid port 426. In the example of FIG. 4B, actuation assembly 428 is illustrated as a push button configured to interface with locking assembly 402 and generate a distal axial force in response to a radial force on the push button; however, in other examples, actuation assembly 428 may include other assemblies configured to generate a rotation force. Pushable actuation assembly 428 includes an actuation housing 436, a push button 430, an intermediate member 432, an extension member 424, and one or more guide members 434.

Extension member 424 is mechanically coupled to one or more therapeutic elements 442 (illustrated as a single therapeutic element 442) at a distal end and fluidically coupled to fluid port 426 at a proximal end, such as described with respect to extension member 324, therapeutic elements 342, and fluid port 326 of FIG. 3B. Extension member 424 is mechanically coupled to intermediate member 432 and configured to axially move within a delivery lumen of elongated body 422 in response to axial movement of intermediate member 432. Intermediate member 432 is configured to translate radial movement of push button 430 into axial movement of extension member 424. As shown, push button 430 has a tapered surface to distally advance intermediate member 432, such as along a track. As a result, radial movement of push button 430 distally advances therapeutic element 342 from therapeutic catheter 420. Intermediate member 432 is also mechanically coupled to guide member 434 and configured to translate radial movement of push button 430 into axial movement of guide member 434, such that guide member 434 may distally extend from housing 436.

FIG. 4C is a side-view cross-sectional conceptual diagram of example medical assembly 440 in an undeployed configuration. Medical assembly 440 may be assembled and positioned such that a distal end 440B may be positioned at a treatment site and a proximal end 440A may be configured to create an interface to translate an actuation force received by actuation assembly 428 to a locking force exerted on therapeutic catheter 420. At distal end 440B, therapeutic catheter 420 may extend from support catheter 400, such that therapeutic element 442 may extend from therapeutic catheter 420 to the treatment site. At proximal end 440A, guide member 434 may be positioned near tapered element 410, such that extension of guide member 434 may cause guide member 434 to contact tapered element 410.

FIG. 4D is a side-view cross-sectional conceptual diagram of the example medical assembly of FIG. 4C in a deployed configuration in accordance with some examples of the present disclosure. To deploy therapeutic element 442, push button 430 may be pressed, such that a tapered surface of push button 430 may engage intermediate member 432 to distally advance intermediate member 432. As a result, extension member 424 may distally advance therapeutic element 442 from elongated body 422 of therapeutic catheter 420. The distal advancement of intermediate member 432 may also exert an axial force on guide member 434 to both distally advance tapered element 410 along a tapered surface of tapered housing 408 and generate a radially inward force on an outer surface of elongated body 422 of therapeutic catheter 420. This radially inward force may fix therapeutic catheter 420 to support catheter 400, such that therapeutic catheter 420 may not move axially within guide lumen 403 of support catheter 400. To withdraw therapeutic element 442, push button 430 may be released, such that extension member 424 may proximally withdraw therapeutic element 442 into therapeutic catheter 420. Tapered element 410 may stop exerting an inward radial force on elongated body 422, such that therapeutic catheter 420 may be moved relative to support catheter 400.

In this way, locking assembly 402 may translate the radial actuation force received by actuation assembly 428 into a radial locking force that secures therapeutic catheter 420 to support catheter 400. In some instances, the radial mechanism of pressable actuation assembly 428 may enable a timing of deployment of therapeutic element 442 and fixation of therapeutic catheter 420 to support catheter 400 to be substantially simultaneous such that timing may be simplified, as radial movement may enable a binary motion. For example, a timing of locking assembly 402 may be substantially simultaneous such that a deployed position of rotational actuation assembly 428 may correspond to both exerting the radially inward force and either extending therapeutic element 442 from therapeutic catheter 420 or therapeutic element 442 contacting a wall of the vessel. Additionally or alternatively, pressable actuation assembly 428 may be configured to provide tactile or audial feedback as to an extent of extension of therapeutic elements 442 and/or whether therapeutic catheter 420 has become fixed to support catheter 400.

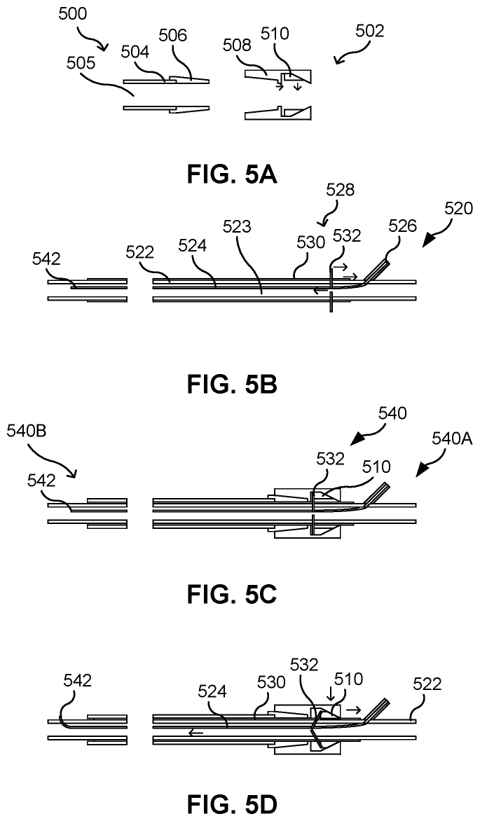

In some examples, the locking assembly is configured to couple to a support catheter and translate an axial actuation force used to operate an actuation assembly of a therapeutic catheter to a radial force to lock the therapeutic catheter to the support catheter. FIGS. 5A-5D describe a medical assembly 540 that includes a support catheter 500, a locking assembly 502 coupled to support catheter 500, and a therapeutic catheter 520 operable by a slidable actuation assembly 528.

FIG. 5A is a side-view cross-sectional conceptual diagram of a proximal portion of support catheter 500 and slidable locking assembly 502 of example medical assembly 540. Support catheter 500 includes an elongated body 504 defining a guide lumen 505 and a proximal connection luer 506, such as described with respect to elongated body 304, guide lumen 305, and connection luer 306 of FIG. 3A. Locking assembly 502 is configured to interface with slidable actuation assembly 528 of therapeutic catheter 520 and translate an axial actuation force received by therapeutic catheter 520 to a radial force. An axial actuation force may include any force generated from axial movement of an actuation element, including derivative forces generated from a combination of axial movement with other structures or surfaces of locking assembly 502 or therapeutic catheter 520.

In the example of FIG. 5A, locking assembly 502 is illustrated as a tapered wedge fitting configured to translate a proximal axial force into an inward radial force; however, in other examples, locking assembly 502 may include other assemblies configured to translate an axial actuation force received by therapeutic catheter 520 into a radial force exerted by support catheter 500. Locking assembly 502 includes a tapered housing 508 that includes a tapered surface and a tapered element 510 configured to advance radially inward in response to an axial force in a proximal direction.

FIG. 5B is a side-view cross-sectional conceptual diagram of therapeutic catheter 520 of example medical assembly 540 having slidable actuation assembly 528. Therapeutic catheter 520 includes actuation assembly 528, an elongated body 522 defining a delivery lumen 523, and a fluid port 526. In the example of FIG. 5B, actuation assembly 528 is illustrated as a slidable sheath configured to interface with locking assembly 502 and generate a proximal axial force in response to a proximal axial force on the slidable sheath; however, in other examples, actuation assembly 528 may include other assemblies configured to generate an axial actuation force. Slidable actuation assembly 528 includes a slidable sheath 530 positioned around elongated body 522, an intermediate member 532, and an extension member 524.

Extension member 524 is mechanically coupled to one or more therapeutic elements (illustrated as a single therapeutic element 542) at a distal end and fluidically coupled to fluid port 526 at a proximal end, such as described with respect to extension member 324, therapeutic elements 342, and fluid port 326 of FIG. 3B. Extension member 524 is mechanically coupled to intermediate member 532 and configured to axially move within delivery lumen 523 of elongated body 522 in response to axial movement of intermediate member 532. Slidable sheath 530 may be configured to move proximally relative to elongated body 522. In the example of FIGS. 5A-5D, intermediate member 532 is configured to translate axial movement of slidable sheath 530 in a proximal direction into axial movement of extension member 524 in a proximal direction outside therapeutic catheter 520 and axial movement of extension member 524 in a distal direction inside therapeutic catheter 520, such as by pivoting at a central fulcrum. As a result, proximal axial movement of slidable sheath 530 distally advances therapeutic element 542 from therapeutic catheter 520, while also proximally advancing tapered element 510 of locking assembly 502.

FIG. 5C is a side-view cross-sectional conceptual diagram of example medical assembly 540 in an undeployed configuration in accordance with some examples of the present disclosure. Medical assembly 540 may be assembled and positioned such that a distal end 540B may be positioned at a treatment site and a proximal end 540A may be configured to create an interface to translate an actuation force received by actuation assembly 528 to a locking force exerted on therapeutic catheter 520. At distal end 540B, therapeutic catheter 520 may extend from support catheter 500, such that therapeutic element 542 may extend from therapeutic catheter 520 to the treatment site. At proximal end 540A, an external portion of intermediate member 532 of actuation assembly 528 may interface with (e.g., contact) a distal surface of tapered element 510 of locking assembly 502.

FIG. 5D is a side-view cross-sectional conceptual diagram of the example medical assembly of FIG. 5C in a deployed configuration in accordance with some examples of the present disclosure. To deploy therapeutic element 542, slidable sheath 530 may be proximally withdrawn, such that intermediate member 532 may engage extension member 524 to distally advance therapeutic element 542 from elongated body 522 of therapeutic catheter 520. The proximal withdrawal of slidable sheath 530 may also exert a proximal axial force on tapered element 510 via intermediate member 532 to proximally withdraw tapered element 510 and generate a radially inward force on an outer surface of elongated body 522 of therapeutic catheter 520. This radially inward force may fix therapeutic catheter 520 to support catheter 500, such that therapeutic catheter 520 may not move axially within guide lumen 503 of support catheter 500. To withdraw therapeutic element 542, slidable sheath 530 may be released or pushed, such that extension member 524 may proximally withdraw therapeutic element 542 into therapeutic catheter 520. Tapered element 510 may stop exerting an inward radial force on elongated body 522, such that therapeutic catheter 520 may be moved relative to support catheter 500.

In this way, locking assembly 502, in combination with actuation assembly 528, may translate the axial actuation force received by actuation assembly 528 into a radial force that secures therapeutic catheter 520 to support catheter 500. In some instances, the axial mechanism of slidable actuation assembly 528 may enable gradations of an extent of advancement or withdrawal. For example, actuation assembly 528 may include tactile gradations, such as ridges, that produces clicks or other tactile or audial feedback indications of the extent of advancement or withdrawal as slidable sheath 530 is moved relative to elongated body 522.

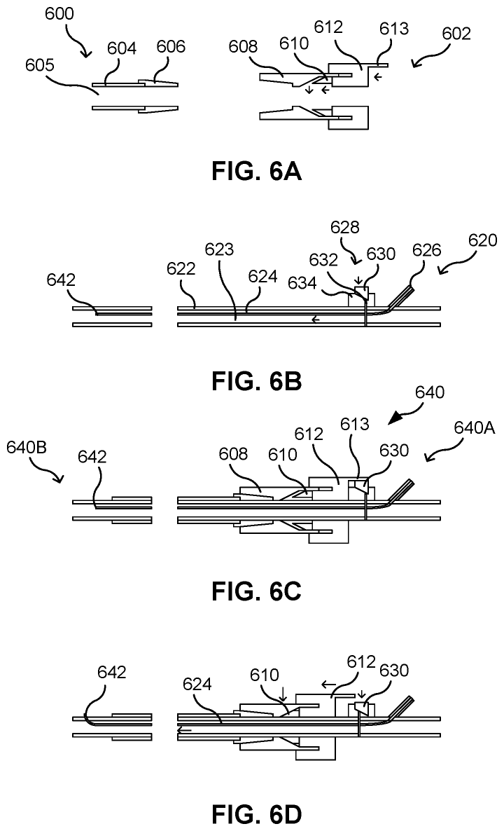

In some examples, such as will be described in FIGS. 6A-6D, a locking assembly is configured to couple to a support catheter and control access to operation of an actuation assembly of a therapeutic catheter by conditioning access of an actuation element of the actuation assembly on generation of a locking force for fixing the therapeutic catheter to the support catheter. For example, a clinician may exert an actuation force on an actuation element of the actuation assembly, such as described in any of FIGS. 3A-3D, 4A-4D, or 5A-5D above. The locking assembly may be configured to restrict access to the actuation element, such as by physically covering or locking the actuation element, until the locking assembly has generated a locking force, such as a radially inward force, on the therapeutic catheter. For example, the same force used to allow access to the actuation element, such as an axial force to physically uncover the actuation element, may be used to generate the locking force. By limiting generation of the actuation force used to deploy the therapeutic elements until after generation of the locking force used to fix the therapeutic catheter to the support catheter, locking mechanisms described herein may ensure that the therapeutic catheter is locked to the support catheter prior to deployment of the therapeutic elements.

In some examples, the locking mechanism is configured to couple to the support catheter, receive an axial access force used to access an actuation assembly of the therapeutic catheter, and translate the axial access force to a radial force to lock the therapeutic catheter to the support catheter. FIGS. 6A-6D describe a medical assembly 640 that includes a support catheter 600, a slidable locking assembly 602 coupled to support catheter 600, and a therapeutic catheter 620 operable by a pushable actuation assembly 628.

FIG. 6A is a side-view cross-sectional conceptual diagram of a support catheter assembly and a slidable locking assembly of an example medical assembly. Support catheter 600 includes an elongated body 604 defining a guide lumen 605 and a proximal connection luer 606, such as described with respect to elongated body 304, guide lumen 305, and connection luer 306 of FIG. 3A. In the example of FIG. 6A, locking assembly 602 is illustrated as a tapered wedge fitting configured to translate a distal axial force into an inward radial force; however, in other examples, locking assembly 602 may include other assemblies configured to translate an axial force received by locking assembly 602 into a radial force exerted by support catheter 600. Locking assembly 602 includes a tapered housing 608 that includes a tapered surface and a tapered element 610 configured to advance radially inward in response to an axial force in a proximal direction. Locking assembly 602 also includes a slidable element 612 configured to interface with tapered housing 608 and axially move relative to housing 608. Slidable element 612 is configured to contact tapered element 610, such that distal axial movement of slidable element 612 generates both distal axial and inward radial movement of slidable element 612. Slidable element 612 includes a cover 613 configured to cover at least a portion of actuation assembly 628.

FIG. 6B is a side-view cross-sectional conceptual diagram of therapeutic catheter 620 of example medical assembly 640 having a pressable actuation assembly 628. Therapeutic catheter 620 includes actuation assembly 628, an elongated body 622 defining a delivery lumen 623, and a fluid port 626. In the example of FIG. 6B, actuation assembly 628 is illustrated as a push button 630; however, in other examples, actuation assembly 428 may include other assemblies. Pushable actuation assembly 628 includes an actuation housing 634, a push button 630, an intermediate member 632, and an extension member 624. Extension member 624 is mechanically coupled to one or more therapeutic elements (illustrated as a single therapeutic element 642) at a distal end and fluidically coupled to fluid port 626 at a proximal end, such as described with respect to extension member 324, therapeutic elements 342, and fluid port 326 of FIG. 3B. Extension member is mechanically coupled to intermediate member 632 and configured to axially move within a delivery lumen of elongated body 622 in response to axial movement of intermediate member 632. Intermediate member 632 is configured to translate radial movement of push button 430 into axial movement of extension member 624. As a result, radial movement of push button 630 distally advances therapeutic element 642 from therapeutic catheter 620.

FIG. 6C is a side-view cross-sectional conceptual diagram of an example medical assembly in an undeployed configuration in accordance with some examples of the present disclosure. Medical assembly 640 may be assembled and positioned such that a distal end 640B may be positioned at a treatment site and a proximal end 640A may be configured to restrict physical access to actuation assembly 628. At distal end 640B, therapeutic catheter 620 may extend from support catheter 600, such that therapeutic element 642 may extend from therapeutic catheter 620 to the treatment site. At proximal end 640A, cover 613 of slidable element 612 may physically cover push button 630 of actuation assembly 628.

FIG. 6D is a side-view cross-sectional conceptual diagram of the example medical assembly of FIG. 6C in a deployed configuration in accordance with some examples of the present disclosure. To access push button 630, slidable element 612 may be distally advanced, such that slidable element 612 may engage tapered element 610 to distally advance tapered element 610 along a tapered surface of tapered housing 608 and generate a radially inward force on an outer surface of elongated body 622 of therapeutic catheter 620. This radially inward force may fix therapeutic catheter 620 to support catheter 600, such that therapeutic catheter 620 may not move axially within guide lumen 605 of support catheter 600. This distal advancement of slidable element 612 may also move cover 613 to expose push button 630. To deploy therapeutic element 642, push button 630 may be pressed, such that a tapered surface of push button 630 may engage intermediate member 632 to distally advance intermediate member 632. As a result, extension member 624 may distally advance therapeutic element 642 from elongated body 622 of therapeutic catheter 620. To withdraw therapeutic element 642, push button 630 may be released, such that extension member 624 may proximally withdraw therapeutic element 642 into therapeutic catheter 620. After release of push button 630, slidable element 612 may be proximally withdrawn, such that tapered element 610 stops exerting an inward radial force on elongated body 622, such that therapeutic catheter 620 may be moved relative to support catheter 600, and cover 613 covers push button 630. In this way, locking assembly 602 may translate the axial access force received by locking assembly 602 into a radial force that secures therapeutic catheter 620 to support catheter 600 and limit access to actuation assembly 628 until therapeutic catheter 620 is secured to support catheter 600. In some instances, support catheter 600 may be used to secure other medical devices delivered through support catheter 600, such as a drug delivery catheter, to support catheter 600 while an actuation assembly of the medical device is accessible.

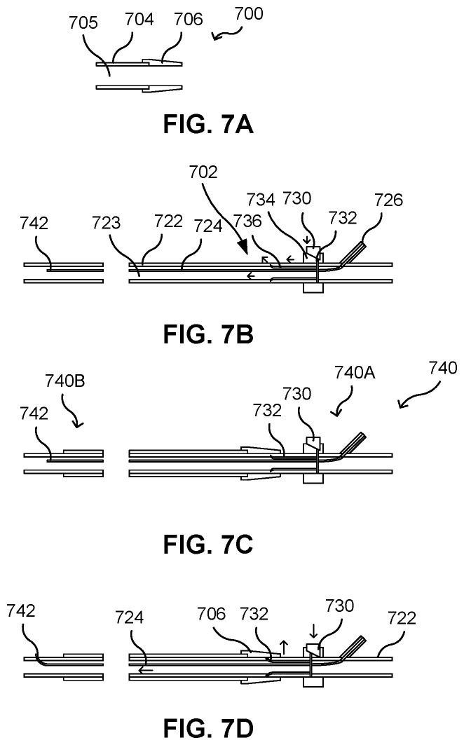

In some examples, such as will be described in FIGS. 7A-7D, the locking assembly is configured to couple to, or be integral with, a therapeutic catheter, rather than a support catheter, interface with an actuation assembly of the therapeutic catheter, and translate an actuation force for operating the actuation assembly into a locking force for fixing the therapeutic catheter to the support catheter. For example, a clinician may exert an actuation force on an actuation element of the actuation assembly to deploy one or more therapeutic elements from the therapeutic catheter. The locking assembly may interface with a portion of the actuation assembly, such as a member translating the actuation force to an extension element for deploying the therapeutic elements, to receive at least a portion of this actuation force and translate the portion of the actuation force into a locking force, such as a radially outward force, on the support catheter. By directly relating the actuation force used to deploy the therapeutic elements to the locking force used to fix the therapeutic catheter to the support catheter, locking mechanisms described herein may ensure that the therapeutic catheter is locked to the support catheter prior to or concurrent with deployment of the therapeutic elements.

FIG. 7A is a side-view cross-sectional conceptual diagram of a support catheter 700 of an example medical assembly 740. Support catheter 700 includes an elongated body 704 defining a guide lumen 705 and a proximal connection luer 706, such as described with respect to elongated body 304, guide lumen 305, and connection luer 306 of FIG. 3A.

FIG. 7B is a side-view cross-sectional conceptual diagram of a locking assembly 702 and a therapeutic catheter 720 of example medical assembly 740 having a pushable actuation assembly 728 in accordance with some examples of the present disclosure. Therapeutic catheter 720 includes actuation assembly 728, an elongated body 722 defining a delivery lumen 723, and a fluid port 726. In the example of FIG. 7B, actuation assembly 728 is illustrated as a push button configured to interface with locking assembly 702 and generate a distal axial force in response to a radial force on the push button; however, in other examples, actuation assembly 728 may include other assemblies, such as actuation assemblies 328 of FIGS. 3A-3D or 528 of FIGS. 5A-5D. Pushable actuation assembly 728 includes an actuation housing 734, a push button 730, an intermediate member 732, and an extension member 724.

Extension member 724 is mechanically coupled to one or more therapeutic elements 742 (illustrated as a single therapeutic element 742) at a distal end and fluidically coupled to fluid port 726 at a proximal end, such as described with respect to extension member 324, therapeutic elements 342, and fluid port 326 of FIG. 3B. Extension member 724 is mechanically coupled to intermediate member 732 and configured to axially move within a delivery lumen of elongated body 722 in response to axial movement of intermediate member 732. Intermediate member 732 is configured to translate radial movement of push button 730 into axial movement of extension member 724. As shown, push button 730 has a tapered surface to distally advance intermediate member 732, such as along a track. As a result, radial movement of push button 730 distally advances therapeutic element 742 from therapeutic catheter 720.

In the example of FIG. 7B, locking assembly 702 includes one or more locking members 736 configured to both move axially within delivery lumen 723 and radially extend through channels or voids in elongated body 722 to contact one or more internal surfaces of support catheter 700. Intermediate member 732 is mechanically coupled to locking members 736 and configured to translate radial movement of push button 730 into axial movement of locking members 736, such that locking members 736 may distally advance through delivery lumen 723 and radially extend from elongated body 722. In other examples, locking assembly 702 may include other mechanisms configured to exert an outward radial force from therapeutic catheter 720 in response to an actuation force on actuation assembly 728. For example, a wall thickness of therapeutic catheter 720 may be locally thin (e.g., patches, bands, or some other configuration) to distend against support catheter 700 and lock against support catheter 700 in response to locking member 736 extending against an inner wall of delivery lumen 723, such that an outside surface of therapeutic catheter 720 may be continuous and provide space for fluids, such as contrast or saline, to be passed through support catheter 700.

FIG. 7C is a side-view cross-sectional conceptual diagram of an example medical assembly in an undeployed configuration in accordance with some examples of the present disclosure. Medical assembly 740 may be assembled and positioned such that a distal end 740B may be positioned at a treatment site and a proximal end 740A may be configured to create an interface to translate an actuation force received by actuation assembly 728 to a locking force exerted on support catheter 700, such that locking member 736 may contact an interior surface of support catheter 700. At distal end 740B, therapeutic catheter 720 may extend from support catheter 700, such that therapeutic element 742 may extend from therapeutic catheter 720 to the treatment site. At proximal end 740A, locking members 736 may be positioned such that extension of locking members 736 may contact at least a portion of support catheter 700, such as an interior surface of guide lumen 705 of elongated body 704 or an interior surface of connection luer 706.

FIG. 7D is a side-view cross-sectional conceptual diagram of the example medical assembly of FIG. 7C in a deployed configuration. To deploy therapeutic element 742, push button 730 may be pressed, such that a tapered surface of push button 730 may engage intermediate member 732 to distally advance intermediate member 732. As a result, extension member 724 may distally advance therapeutic element 742 from elongated body 722 of therapeutic catheter 720. The distal advancement of intermediate member 732 may also exert an axial force on locking members 736 to both distally advance locking members 736 within delivery lumen 723 and extend locking members 736 from elongated body 722 to exert a radially outward force on an inner surface of support catheter 700. This radially outward force may fix therapeutic catheter 720 to support catheter 700, such that therapeutic catheter 720 may not move axially within guide lumen 705 of support catheter 700. To withdraw therapeutic element 742, push button 730 may be released, such that extension member 724 may proximally withdraw both therapeutic element 742 and locking members 736 into therapeutic catheter 720, such that therapeutic catheter 720 may be moved relative to support catheter 700.

In this way, locking assembly 702 may translate the actuation force received by actuation assembly 728 into a radial locking force that secures therapeutic catheter 720 to support catheter 400. In some instances, integration or coupling of locking assembly 702 to therapeutic catheter 720 may enable a relative difference in position of therapeutic catheter 720 and support catheter 700, such that a distal end of therapeutic catheter 720 may be positioned at a variety of positions at the treatment site without moving support catheter 700. For example, locking assembly 702 may not require a specific interface between therapeutic catheter 720 and support catheter 700, and may contact support catheter 700 at a variety of locations to secure therapeutic catheter 720 to support catheter 700.

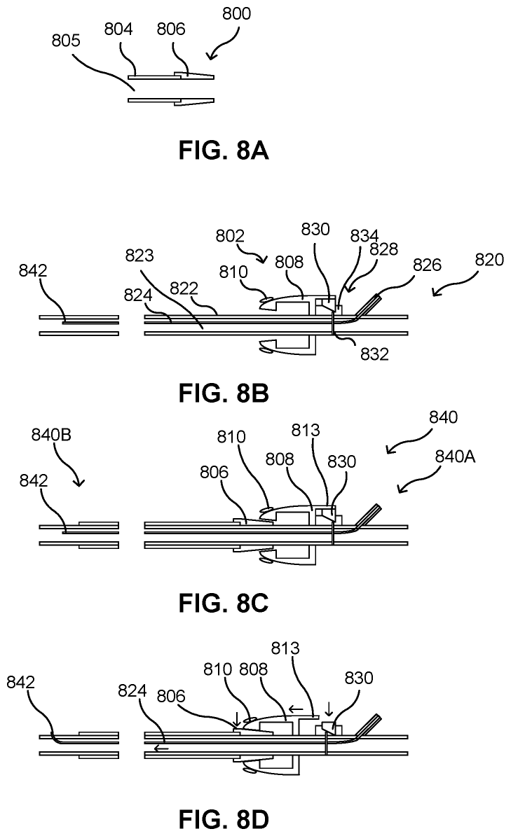

In some examples, such as will be described in FIGS. 8A-8D, the locking assembly is configured to couple to, or be integral with, a therapeutic catheter and control access to operation of an actuation assembly of the therapeutic catheter by conditioning access of an actuation element of the actuation assembly on generation of a locking force for fixing the therapeutic catheter to a support catheter. For example, a clinician may exert an actuation force on an actuation element of the actuation assembly, such as described in any of FIGS. 3A-3D, 4A-4D, or 5A-5D above. The locking assembly may be configured to restrict access to the actuation element, such as by physically covering or locking the actuation element, until the locking assembly has generated a locking force, such as a radially inward force, on the support catheter. For example, the same force used to allow access to the actuation element, such as an axial force to physically uncover the actuation element, may be used to generate the locking force. By limiting generation of the actuation force used to deploy the therapeutic elements until after generation of the locking force used to fix the therapeutic catheter to the support catheter, locking mechanisms described herein may ensure that the therapeutic catheter is locked to the support catheter prior to deployment of the therapeutic elements.

FIG. 8A is a side-view cross-sectional conceptual diagram of a support catheter 800 of an example medical assembly in accordance with some examples of the present disclosure. Support catheter 800 includes an elongated body 804 defining a guide lumen 805 and a proximal connection luer 806, such as described with respect to elongated body 304, guide lumen 305, and connection luer 306 of FIG. 3A.

FIG. 8B is a side-view cross-sectional conceptual diagram of a slidable locking assembly 802 and a therapeutic catheter 820 of an example medical assembly 840 having a pressable actuation assembly 828. Therapeutic catheter 820 includes actuation assembly 828, an elongated body 822 defining a delivery lumen 823, and a fluid port 826. In the example of FIG. 8A, actuation assembly 828 is illustrated as a push button 830; however, in other examples, actuation assembly 828 may include other assemblies, such as actuation assemblies 328 of FIGS. 3A-3D or 528 of FIGS. 5A-5D. Pushable actuation assembly 828 includes an actuation housing 834, a push button 830, an intermediate member 832, and an extension member 824, and may be similarly operable to pushable actuation assembly 628 of FIG. 6B. Extension member 824 is mechanically coupled to one or more therapeutic elements (illustrated as a single therapeutic element 842) at a distal end and fluidically coupled to fluid port 826 at a proximal end, such as described with respect to extension member 324, therapeutic elements 342, and fluid port 326 of FIG. 3B.

In the example of FIG. 8B, locking assembly 802 is illustrated as a slidable tension fitting configured to translate a distal axial force into an inward radial force; however, in other examples, locking assembly 802 may include other assemblies configured to translate an axial force received by locking assembly 802 into a radial force exerted on support catheter 800. Locking assembly 802 also includes a slidable housing 808 configured to axially move relative to elongated body 822 and contact support catheter 800. Slidable housing 808 includes a cover 813 configured to cover at least a portion of actuation assembly 828. Locking assembly 802 includes a tension element 810 positioned around slidable housing 808 and configured to exert a radially inward force on a portion of locking housing 808 contacting support catheter 800. A tension of tension element 810 may be configured to provide a desired amount of inward radial force on support catheter 800, such that therapeutic catheter 820 may remain fixed to support catheter 800 during deployment of therapeutic elements 842.

FIG. 8C is a side-view cross-sectional conceptual diagram of example medical assembly 840 in an undeployed configuration. Medical assembly 840 may be assembled and positioned such that a distal end 840B may be positioned at a treatment site and a proximal end 840A may be configured to restrict physical access to actuation assembly 828. At distal end 840B, therapeutic catheter 820 may extend from support catheter 800, such that therapeutic element 842 may extend from therapeutic catheter 820 to the treatment site. At proximal end 840A, cover 813 of slidable housing 808 may physically cover push button 830 of actuation assembly 828, and may be positioned sufficiently close to support catheter 800 that distal advancement of slidable housing 808 may contact support catheter 800.

FIG. 8D is a side-view cross-sectional conceptual diagram of the example medical assembly 840 of FIG. 8C in a deployed configuration. To access push button 830, slidable housing 808 may be distally advanced, such that a distal portion of slidable housing 808 may engage support catheter 800 and generate, via tension element 810, a radially inward force on an outer surface of support catheter 800. This radially inward force may fix therapeutic catheter 820 to support catheter 800, such that therapeutic catheter 820 may not move axially within guide lumen 805 of support catheter 800. This distal advancement of slidable housing 808 may also move cover 813 to expose push button 830. To deploy therapeutic element 842, push button 830 may be pressed, such that a tapered surface of push button 830 may engage intermediate member 832 to distally advance intermediate member 832. As a result, extension member 824 may distally advance therapeutic element 842 from elongated body 822 of therapeutic catheter 820. To withdraw therapeutic element 842, push button 830 may be released, such that extension member 824 may proximally withdraw therapeutic element 842 into therapeutic catheter 820. After release of push button 830, slidable housing 808 may be proximally withdrawn, such that tension element 810 stops exerting an inward radial force on support catheter 800, such that therapeutic catheter 820 may be moved relative to support catheter 800, and cover 813 covers push button 830.

In this way, locking assembly 802 may translate the axial access force received by locking assembly 802 into a radial force that secures therapeutic catheter 820 to support catheter 800 and limit access to actuation assembly 828 until therapeutic catheter 820 is secured to support catheter 800. In some instances, locking assembly 802 may be customized for an existing therapeutic catheter 820, such that therapeutic catheter 820 may be fitted with locking assembly 802. For example, locking housing 808, tension element 810, and cover 813 may be configured to move relative to a particular diameter of elongated body 822, secure to a particular diameter or range of diameters of elongated body 804 of support catheter 800, and cover a particular actuation assembly 828.

While the medical assemblies illustrated in FIGS. 3A-3D, 4A-4D, 5A-5D, 6A-6D, 7A-7D, and 8A-8D are described with respect to delivery of a chemical agent through penetrating needles, any of the medical assemblies may be used for other treatment modalities, such as delivery of RF energy or delivery of a chemical agent through other means. Additionally, while the medical assemblies illustrated in FIGS. 3A-3D, 4A-4D, 5A-5D, 6A-6D, 7A-7D, and 8A-8D are described with respect to delivery of therapy through therapeutic elements that extend from a therapeutic catheter, any of the medical assemblies may be used to control delivery of energy or a therapeutic agent from one or more therapeutic elements, rather than or in addition to controlling extension of the therapeutic elements.

In some examples, the locking assembly may be configured to fix the therapeutic catheter relative to the support catheter while the therapeutic catheter delivers a chemical agent to the tissues without penetrating a wall of a vessel, such as by using one or more apertures. The apertures may be fluidically coupled to a pressurized fluid source and configured to position within a lumen of a vessel and deliver the chemical agent to the tissue, such as by discharging fluid jets of the chemical agent to a target treatment site at the wall of the vessel, such that the chemical agent may penetrate the wall of the vessel into the tissues. A clinician may exert an actuation force on an actuation element of an actuation assembly to deliver a chemical agent under pressure through the apertures to the target treatment site. The locking assembly may be configured to prevent delivery of pressurized chemical agent, such as by blocking fluid flow, controlling the fluid source, or restricting access to the actuation element, until the locking assembly has generated a locking force, such as a radially inward or outward force, on the support catheter or therapeutic catheter. By limiting delivery of the chemical agent until after generation of the locking force used to fix the therapeutic catheter to the support catheter, locking mechanisms described herein may ensure that the therapeutic catheter is locked to the support catheter during delivery of the chemical agent.

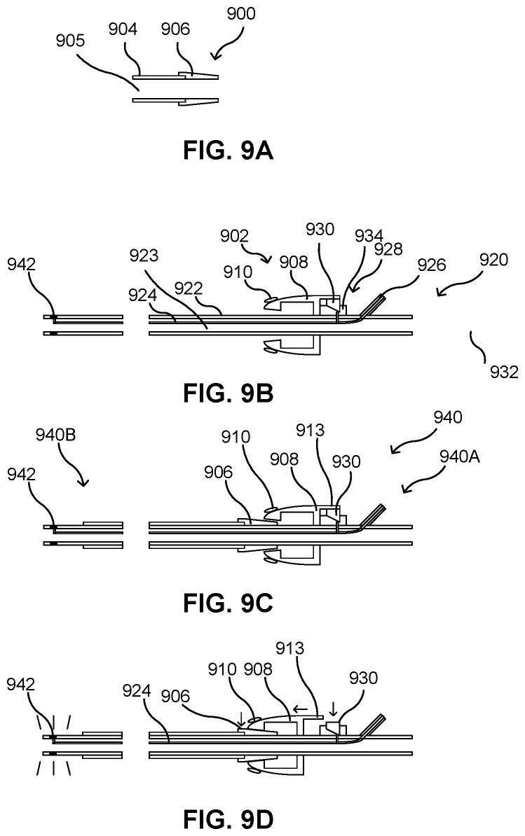

In some examples, such as illustrated in FIGS. 9A-9D, the locking assembly may be configured to fix the therapeutic catheter relative to the support catheter while the therapeutic catheter delivers energy to the tissues using fixed electrodes. For example, a clinician may exert an actuation force on an actuation element of the actuation assembly to deliver current to the electrodes. The locking assembly may be configured to prevent delivery of current to the electrodes, such as by blocking current, controlling a current source, or restricting access to the actuation element, until the locking assembly has generated a locking force, such as a radially inward or outward force, on the support catheter or therapeutic catheter. By limiting delivery of the energy or therapeutic agent until after generation of the locking force used to fix the therapeutic catheter to the support catheter, locking mechanisms described herein may ensure that the therapeutic catheter is locked to the support catheter during delivery of the energy or therapeutic agent.

FIG. 9A is a side-view cross-sectional conceptual diagram of a support catheter 900 of an example medical assembly in accordance with some examples of the present disclosure. Support catheter 900 includes an elongated body 904 defining a guide lumen 905 and a proximal connection luer 906, such as described with respect to elongated body 304, guide lumen 305, and connection luer 306 of FIG. 3A.

FIG. 9B is a side-view cross-sectional conceptual diagram of a slidable locking assembly 902 and a therapeutic catheter 920 of an example medical assembly 940 having a pressable actuation assembly 928. Therapeutic catheter 920 includes actuation assembly 928, an elongated body 922 defining a delivery lumen 923, and an electrical connector 926 configured to connect to a current source; however, in other examples, elongated body 922 may include an insulative material, rather than delivery lumen 923. In the example of FIG. 9A, actuation assembly 928 is illustrated as a push button 930; however, in other examples, actuation assembly 928 may include other assemblies, such as actuation assemblies 928 of FIGS. 3A-3D or 928 of FIGS. 5A-5D. Pushable actuation assembly 928 includes an actuation housing 934, a push button 930, an electrical switch 932, and an electrical conductor 924. Electrical conductor 924 is electrically coupled to one or more therapeutic elements (illustrated as one therapeutic element 942 configured as a ring electrode) at a distal end and selectively electrically coupled to electrical connector 926 at a proximal end. When pushed, push button 930 may operate electrical switch 932 to permit current to flow from electrical connector 926 to therapeutic elements 942 via electrical conductor 924.

In the example of FIG. 9B, locking assembly 902 is illustrated as a slidable tension fitting configured to translate a distal axial force into an inward radial force; however, in other examples, locking assembly 902 may include other assemblies configured to translate an axial force received by locking assembly 902 into a radial force exerted on support catheter 900. Locking assembly 902 includes a slidable housing 908 configured to axially move relative to elongated body 922 and contact support catheter 900. Slidable housing 908 includes a cover 913 configured to cover at least a portion of actuation assembly 928. Locking assembly 902 includes a tension element 910 positioned around slidable housing 908 and configured to exert a radially inward force on a portion of locking housing 908 contacting support catheter 900. A tension of tension element 910 may be configured to provide a desired amount of inward radial force on support catheter 900, such that therapeutic catheter 920 may remain fixed to support catheter 900 during delivery of energy from therapeutic elements 942.

FIG. 9C is a side-view cross-sectional conceptual diagram of example medical assembly 940 in an inactivated configuration. Medical assembly 940 may be assembled and positioned such that a distal end 940B may be positioned at a treatment site and a proximal end 940A may be configured to restrict physical access to actuation assembly 928. At distal end 940B, therapeutic catheter 920 may extend from support catheter 900, such that therapeutic element 942 may extend from therapeutic catheter 920 to the treatment site. At proximal end 940A, cover 913 of slidable housing 908 may physically cover push button 930 of actuation assembly 928, and may be positioned sufficiently close to support catheter 900 that distal advancement of slidable housing 908 may contact support catheter 900.

FIG. 9D is a side-view cross-sectional conceptual diagram of the example medical assembly 940 of FIG. 9C in an activated configuration delivering energy to a tissue. To access push button 930, slidable housing 908 may be distally advanced, such that a distal portion of slidable housing 908 may engage support catheter 900 and generate, via tension element 910, a radially inward force on an outer surface of support catheter 900. This radially inward force may fix therapeutic catheter 920 to support catheter 900, such that therapeutic catheter 920 may not move axially within guide lumen 905 of support catheter 900. This distal advancement of slidable housing 908 may also move cover 913 to expose push button 930. To deliver current to therapeutic element 942, push button 930 may be pressed, such that push button 930 may operate switch 932 to electrically couple electrical conductor 924 to electrical connector 926. As a result, electrical conductor 924 may deliver current to therapeutic elements 942. To stop delivering current to therapeutic elements 942, push button 930 may be released, such that switch 932 may cease to electrically couple electrical connector 926 to electrical conductor 924. After release of push button 930, slidable housing 908 may be proximally withdrawn, such that tension element 910 stops exerting an inward radial force on support catheter 900, such that therapeutic catheter 920 may be moved relative to support catheter 900, and cover 913 covers push button 930. In this way, locking assembly 902 may translate the axial access force received by locking assembly 902 into a radial force that secures therapeutic catheter 920 to support catheter 900 and limit access to actuation assembly 928 until therapeutic catheter 920 is secured to support catheter 900.

While FIG. 9A-9D has been described with respect to slidable locking assembly 928 coupled to therapeutic catheter 920, in other examples, locking assemblies may use other mechanisms to fix therapeutic catheter 920 relative to support catheter 900 while therapeutic elements 942 deliver therapy, such as energy or a chemical agent, to the tissues. For example, any of therapeutic catheters 320, 420, 520, 620, 720, or 820, may be configured to deliver therapy, such as RF energy, from therapeutic elements, such that any of locking assemblies 328, 428, 528, 628, 728, or 828 may be configured fix a corresponding therapeutic catheter to a corresponding support catheter while delivering therapy to the tissue.

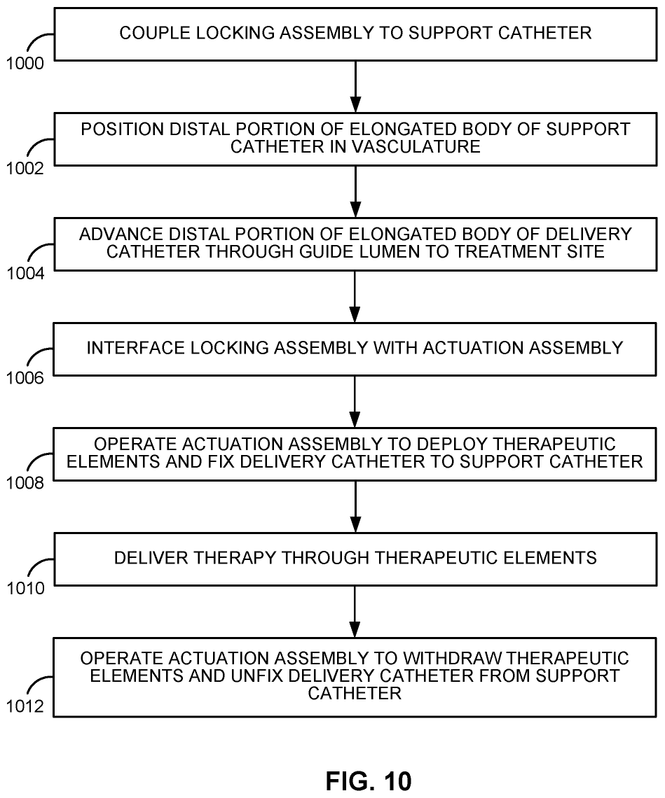

FIG. 10 is a flow diagram illustrating an example technique of positioning and deploying therapeutic elements for neuromodulation of nerves with an interlock between securing a medical assembly and deploying the therapeutic elements, in accordance with some examples of this disclosure. The technique of FIG. 10 will be described with concurrent reference to medical assembly 340 of FIGS. 3A-3D, although it will be appreciated that the technique of FIG. 10 may be performed with other medical assemblies, such as other medical assemblies described herein. Conversely, it will also be appreciated that medical assembly 340 of FIG. 3 may be used in other techniques.

The technique of FIG. 10 includes coupling locking assembly 302 to support catheter 300. For example, a clinician may position connection luer 306 of support catheter 300 into a receptacle of threaded housing 308 (1000). In some instances, connection luer 306 may be configured to couple to different portions on elongated body of support catheter 300. The technique of FIG. 10 includes positioning a distal portion of elongated body 304 of support catheter 300 in a renal vessel of a patient (1002). A clinician may access the renal vessel of the patient through an intravascular path, such as a percutaneous access site in the femoral, brachial, radial, or axillary artery to a targeted treatment site within the renal vessel. By manipulating a proximal portion of support catheter 300 from outside the intravascular path, a clinician may advance at least the distal portion through the sometimes tortuous intravascular path and remotely manipulate the distal portion. The technique of FIG. 10 includes advancing a distal portion of therapeutic catheter 320 through guide lumen 305 of elongated body 304 of support catheter 300 (1004). The clinician may manipulate a proximal portion of therapeutic catheter 320, such as a handle, so that a distal end of elongated body 304 that includes therapeutic elements 342 is positioned near a treatment site.

The technique of FIG. 10 includes interfacing locking assembly 302 with actuation assembly 328 of therapeutic catheter 320 (1006). Therapeutic catheter 320 may be advanced until actuation assembly 328 and locking assembly 302 are positioned such that rotation of housing 330 may thread onto housing 308 and axially advance housing 330 relative to housing 308. As a result, a rotation actuation force applied to housing 330 may translate into an axial force on compression sleeve 310.

The technique of FIG. 10 includes operating actuation assembly 328 to deploy therapeutic elements 342 and fix therapeutic catheter 320 relative to support catheter 300 (1008). For example, the clinician may rotate housing 330 to distally advance housing 330. This distal advancement may cause extension member 324 to distally advance and extend therapeutic elements 342 from therapeutic catheter 320 to deploy to the treatment site. Therapeutic elements 342 may at least partially penetrate a wall of a vessel at the treatment site, such that tissues near the wall of the vessel may receive a chemical agent from therapeutic elements 342. Additionally, the distal advancement of housing 330 causes compression sleeve 310 to compress elastic sleeve 312 to exert an inward radial force on elongated body 322 of therapeutic catheter 320 to fix therapeutic catheter 320 to support catheter 300. As a result, the clinician may not move therapeutic catheter 320 relative to support catheter 300 while therapeutic elements 342 are deployed, thereby protecting the wall of the vessel.

The technique of FIG. 10 includes delivery therapy through therapeutic elements 342 (1010), such as by injecting a chemical agent through therapeutic elements 342. For example, the clinician may connect a chemical agent source to port 326 and deliver the chemical agent from port 326 through therapeutic elements 342 to modulate activity of at least one renal nerve adjacent to the vessel. During this injection, movement of therapeutic catheter 320 relative to support catheter 300, such as movement caused by clenching of the patient, may be reduced due to fixation of therapeutic catheter 320 to support catheter 300.

The technique of FIG. 10 includes operating actuation assembly 328 to withdraw therapeutic elements 342 into therapeutic catheter 320 and unfix therapeutic catheter 320 from support catheter 300 (1012). For example, the clinician may rotate housing 308 in an opposite direction as used to deploy therapeutic elements 342 to axially withdrawn housing 308, thereby proximally withdrawing extension member 324 and uncompressing elastic sleeve 312. As a result, therapeutic elements 342 may be withdrawn into therapeutic catheter 320 and an inward radial force may no longer be exerted on elongated body 322 by locking assembly 302. The clinician may remove therapeutic catheter 320 from support catheter 300.

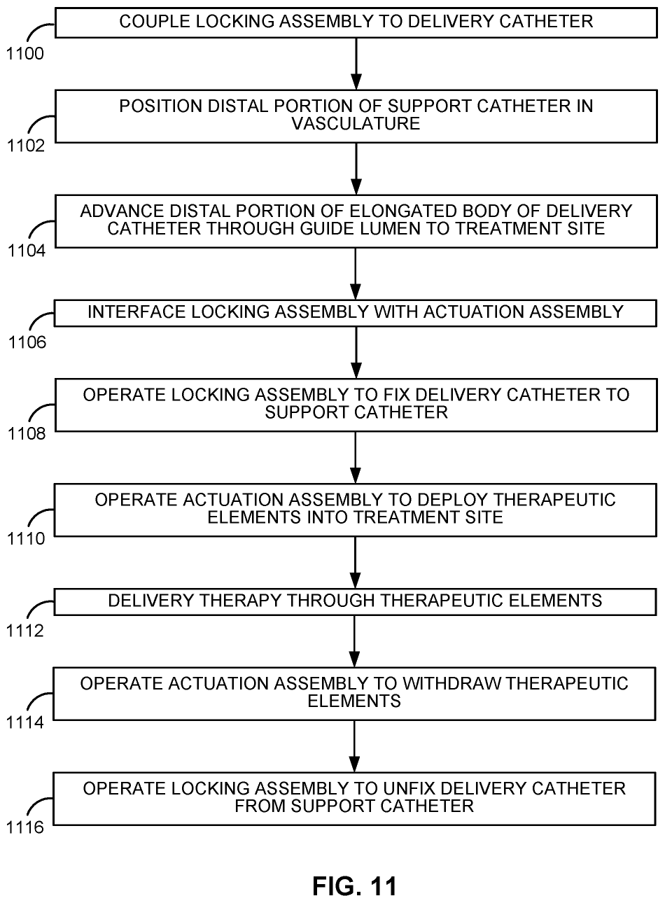

FIG. 11 is a flow diagram illustrating an example technique of positioning and deploying therapeutic elements for neuromodulation of nerves with a forced order of operations of securing a medical assembly and subsequently deploying the therapeutic elements, in accordance with some examples of this disclosure. The technique of FIG. 11 will be described with concurrent reference to medical assembly 840 of FIGS. 8A-8D, although it will be appreciated that the technique of FIG. 11 may be performed with other medical assemblies, such as other medical assemblies described herein. Conversely, it will also be appreciated that medical assembly 840 of FIG. 8 may be used in other techniques.

The technique of FIG. 11 includes coupling locking assembly 802 to therapeutic catheter 820 (1100). For example, a clinician may position locking assembly 802 on therapeutic catheter 820. The technique of FIG. 10 includes positioning a distal portion of elongated body 804 of support catheter 800 in a renal vessel of a patient (1102) and advancing a distal portion of therapeutic catheter 820 through guide lumen 805 of elongated body 804 of support catheter 800 (1104), such as described in steps 1002 and 1004 of FIG. 10.

The technique of FIG. 11 includes interfacing locking assembly 802 with actuation assembly 828 of therapeutic catheter 820 and a portion of support catheter 800 (1106). Locking assembly 802 may be proximally withdrawn until actuation assembly cover 813 of slidable housing 808 covers push button 830 and at least a portion of support catheter 800 is close enough to locking assembly 802, such that slidable housing 808 may contact and secure to support catheter 800 when operated. As a result, an axial actuation force applied to slidable housing 808 may translate into a radial force on support catheter 800.

The technique of FIG. 11 includes operating locking assembly 802 to fix therapeutic catheter 820 to support catheter 800 (1108). For example, the clinician may distally advance slidable housing 808 over a portion of support catheter 800. This distal advancement may cause a distal portion of slidable housing 808 to an exterior surface of support catheter 800. Tension element 810 may exert a radially inward force on support catheter 800 via slidable housing 808, such that slidable housing 808, and therefore therapeutic catheter 820 may be secured to support catheter 800. As a result, the clinician may not move therapeutic catheter 820 relative to support catheter 800 while actuation assembly 828 is accessible for operation, thereby protecting the wall of the vessel.

The technique of FIG. 11 includes operating actuation assembly 828 to deploy therapeutic elements 842 (1110). For example, the clinician may press push button 830 to cause extension member 824 to distally advance and extend therapeutic elements 842 from therapeutic catheter 820 to deploy to the treatment site. Therapeutic elements 842 may at least partially penetrate a wall of a vessel at the treatment site, such that tissues near the wall of the vessel may receive a chemical agent from therapeutic elements 842. The technique of FIG. 10 includes delivering therapy through therapeutic elements 842 (1112), such as described in step 1010 of FIG. 10.

The technique of FIG. 11 includes operating actuation assembly 828 to withdraw therapeutic elements 842 into therapeutic catheter 820 (1114). For example, the clinician may release push button 830 to proximally withdraw extension member 824. As a result, therapeutic elements 842 may be withdrawn into therapeutic catheter 820. The technique of FIG. 11 includes operating locking assembly 802 to unfix therapeutic catheter 820 from support catheter 300 (1116). For example, the clinician may proximally withdraw slidable housing 808 to remove a distal portion of slidable housing 808 from contacting support catheter 800 and exerting an inward radial force on support catheter 800 and physically restrict access to push button 830. The clinician may remove therapeutic catheter 820 from support catheter 800.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure. For example, while particular features of the neuromodulation catheters were described as being part of a single device, in other examples, these features can be included on one or more separate devices that can be positioned adjacent to and/or used in tandem with the neuromodulation catheters to perform similar functions to those described herein. Additionally, while the description of the present technology is focused on therapeutic elements that deliver chemical agents, the present technology can equally be applied to therapeutic elements for delivering other methods of neuromodulation therapy, including cooling, heating, electrical stimulation (using needle electrodes), RF energy delivery (using needle electrodes), microwave energy delivery (using microwave needles), ultrasound (using ultrasound transducers), or the like.

Certain aspects of the present disclosure described in the context of particular examples may be combined or eliminated in other embodiments. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within the single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique, or any combination thereof.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “about” or “approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

Aspects and embodiments of the invention may be defined by the following clauses.

Clause 1. A medical assembly, comprising:
a support catheter comprising an elongated body defining a guide lumen and configured to position directly within vasculature of a patient;
a therapeutic catheter comprising:
an elongated body defining a delivery lumen and configured to move within the guide lumen;
one or more therapeutic elements at a distal portion of the elongated body, wherein the one or more therapeutic elements are configured to extend from the delivery lumen into a deployed configuration at a treatment site of the patient; and
an actuation assembly configured to deploy the one or more therapeutic elements to extend from the delivery lumen into the deployed configuration; and
a locking assembly coupled to at least one of the support catheter or the therapeutic catheter, wherein the locking assembly is configured to fix the therapeutic catheter relative to the support catheter while the one or more therapeutic elements are extended from the delivery lumen.

Clause 2. The medical assembly of clause 1, wherein the locking assembly fixes the therapeutic catheter relative to the support catheter prior to the one or more therapeutic elements extending from the delivery lumen to the deployed configuration.

Clause 3. The medical assembly of clause 1 or 2, wherein the locking assembly fixes the therapeutic catheter relative to the support catheter prior to the one or more therapeutic elements extending from the delivery lumen.

Clause 4. The medical assembly of any of clauses 1 to 3, wherein the locking assembly fixes the therapeutic catheter relative to the support catheter until the one or more therapeutic elements retract into the delivery lumen to a delivery configuration.

Clause 5. The medical assembly of any of clauses 1 to 4, wherein the locking assembly fixes the therapeutic catheter relative to the support catheter until the one or more therapeutic elements retract into the delivery lumen.

Clause 6. The medical assembly of any of clauses 1 to 5,
wherein the locking assembly is coupled to the support catheter, and
wherein the locking assembly is configured to exert a radially inward force on an outer surface of the elongated body of the therapeutic catheter to fix the therapeutic catheter relative to the support catheter.

Clause 7. The medical assembly of any of clauses 1 to 5,
wherein the locking assembly is coupled to the therapeutic catheter, and
wherein the locking assembly is configured to exert a radially outward force on an inner surface of the elongated body of the support catheter to fix the therapeutic catheter relative to the support catheter.

Clause 8. The medical assembly of any of clauses 1 to 7, wherein the locking assembly and the actuation assembly are configured to receive an actuation force and translate the actuation force into a locking force to fix the therapeutic catheter relative to the support catheter.

Clause 9. The medical assembly of any of clauses 1 to 8, further comprising an actuation element configured to:
operate the locking assembly to fix the therapeutic catheter relative to the support catheter; and
operate the actuation assembly to deploy the one or more therapeutic elements.

Clause 10. The medical assembly of clause 9, wherein the actuation element is configured to simultaneously:
operate the locking assembly to fix the therapeutic catheter relative to the support catheter; and
operate the actuation assembly to extend the one or more therapeutic elements from the delivery lumen into the deployed configuration.

Clause 11. The medical assembly of clause 9, wherein the actuation element is configured to:
at a first position, operate the locking assembly to fix the therapeutic catheter relative to the support catheter; and
at a second position, operate the actuation assembly to extend the one or more therapeutic elements from the delivery lumen into the deployed configuration.

Clause 12. The medical assembly of any of clauses 9 to 11, wherein the actuation element comprises a pressable button configured to move between a locked position and an unlocked position.

Clause 13. The medical assembly of any of clauses 9 to 11, wherein the actuation element comprises a rotatable knob configured to rotate between a locked position and an unlocked position.

Clause 14. The medical assembly of any of clauses 9 to 11, wherein the actuation element comprises a slidable sheath configured to move between a locked position and an unlocked position.

Clause 15. The medical assembly of any of clauses 1 to 7, wherein the locking assembly is configured to permit operation of the actuation assembly.

Clause 16. The medical assembly of clause 15, wherein the locking assembly further comprises a movable element configured to:
move between a locked position and an unlocked position;
when in the locked position, permit operation of the actuation assembly by exposing an actuation element configured to operate the actuation assembly, and
when in the unlocked position, permit movement of the therapeutic catheter relative to the support catheter.

Clause 17. The medical assembly of any of clauses 1 to 16,
wherein each of the one or more therapeutic elements includes a needle, and
wherein, in a deployed configuration, each needle of the one or more therapeutic elements is configured to extend at least partially through a wall of the vasculature at the treatment site.

Clause 18. The medical assembly of clause 17, wherein the one or more therapeutic elements are each configured to deliver a chemical agent to the treatment site.

Clause 19. The medical assembly of clause 18, wherein the chemical agent is configured to modulate activity of at least one renal nerve in perivascular tissue.

Clause 20. The medical assembly of any of clauses 17 to 19,
wherein each of the one or more therapeutic elements further comprises a guide tube, and
wherein the needle of each therapeutic element is configured to be moved relative to the corresponding guide tube.

Clause 21. The medical assembly of any of clauses 1 to 20, wherein the therapeutic catheter is an ablation catheter.

Clause 22. The medical assembly of any of clauses 1 to 21, wherein each of the one or more therapeutic elements comprises an electrode.

Clause 23. A method comprising:
positioning a distal portion of an elongated body of a support catheter in vasculature of a patient, wherein the elongated body of the support catheter defines a guide lumen;
advancing a distal portion of an elongated body of a therapeutic catheter through the guide lumen to a treatment site of the patient, wherein the elongated body defines a delivery lumen, and wherein the distal portion of the therapeutic catheter comprises one or more therapeutic elements configured to extend from the delivery lumen into a deployed configuration at the treatment site;
fixing the therapeutic catheter relative to the support catheter; and
deploying the one or more therapeutic elements to extend from the delivery lumen into the deployed configuration.

Clause 24. The method of clause 23, wherein the locking assembly fixes the therapeutic catheter relative to the support catheter prior to one or more therapeutic elements extending from the delivery lumen to the deployed configuration.

Clause 25. The method of clause 23 or 24, wherein the locking assembly fixes the therapeutic catheter relative to the support catheter prior to the one or more therapeutic elements extending from the delivery lumen.

Clause 26. The method of any of clauses 23 to 25, wherein the locking assembly fixes the therapeutic catheter relative to the support catheter until the one or more therapeutic elements retract into the delivery lumen to a delivery configuration.

Clause 27. The method of any of clauses 23 to 26, wherein the locking assembly fixes the therapeutic catheter relative to the support catheter until the one or more therapeutic elements retract into the delivery lumen.

Clause 28. The method of any of clauses 23 to 27, further comprising:
operating an actuation assembly to deploy the one or more therapeutic elements; and
operating a locking assembly to fix the therapeutic catheter relative to the support catheter.

Clause 29. The method of clause 28,
wherein, prior to operation, the locking assembly is coupled to the support catheter, and
wherein operating the locking assembly comprises exerting a radially inward force on an outer surface of the elongated body of the therapeutic catheter to fix the therapeutic catheter relative to the support catheter.

Clause 30. The method of clause 28,
wherein, prior to operation, the locking assembly is coupled to the therapeutic catheter, and
wherein operating the locking assembly comprises exerting a radially outward force on an inner surface of the elongated body of the support catheter to fix the therapeutic catheter relative to the support catheter.

Clause 31. The method of any of clauses 28 to 30, further comprising operating an actuation element to operate the actuation assembly and the locking assembly.

Clause 32. The method of clause 31, wherein operating the actuation element comprises simultaneously:
operating the locking assembly to fix the therapeutic catheter relative to the support catheter; and
operating the actuation assembly to extend the one or more therapeutic elements from the delivery lumen into the deployed configuration.

Clause 33. The method of clause 31, wherein operating the actuation element comprises:
at a first position, operating the locking assembly to fix the therapeutic catheter relative to the support catheter; and
at a second position, operating the actuation assembly to extend the one or more therapeutic elements from the delivery lumen into the deployed configuration.

Clause 34. The method of any of clauses 31 to 33, wherein the actuation element comprises a pressable button.

Clause 35. The method of any of clauses 31 to 33, wherein the actuation element comprises a rotatable knob.

Clause 36. The method of any of clauses 31 to 33, wherein the actuation element comprises a slidable sheath.

Clause 37. The method of any of any of clauses 28 to 30, wherein operating the locking assembly permits operation of the actuation assembly.

Clause 38. The method of clause 37, wherein operating the locking assembly further comprises:
moving a movable element from an unlocked position to a locked position to fix the therapeutic catheter relative to the support catheter and expose an actuation element; and
operating the actuation element to operate the actuation assembly.

Clause 39. The method of any of clauses 23 to 38,
wherein each of the one or more therapeutic elements includes a needle, and
wherein, in a deployed configuration, each needle of the one or more therapeutic elements are configured to extend at least partially through a wall of the vasculature at the treatment site.

Clause 40. The method of clause 39, further comprising delivering a chemical agent through the one or more therapeutic elements to the treatment site.

Clause 41. The method of clause 40,
wherein the treatment site includes perivascular tissue of a renal artery, and
wherein the chemical agent is delivered to modulate activity of at least one renal nerve in the perivascular tissue.

Clause 42. The method of clause 40 or 41, wherein the chemical agent comprises an alcohol.

Clause 43. The method of any of clauses 39 to 42, wherein each of the one or more therapeutic elements further comprises a guide tube, and wherein the needle of each therapeutic element is configured to be moved relative to the corresponding guide tube.

Clause 44. A therapeutic catheter comprising:
an elongated body defining a delivery lumen and configured to move within a guide lumen of a support catheter;
one or more therapeutic elements at a distal portion of the elongated body, wherein the one or more therapeutic elements are configured to extend from the delivery lumen into a deployed configuration at a treatment site of the patient;
an actuation assembly configured to deploy the one or more therapeutic elements to extend from the delivery lumen into the deployed configuration; and
a locking assembly configured to fix the therapeutic catheter relative to the support catheter while the one or more therapeutic elements are extended from the delivery lumen.

Clause 45. The therapeutic catheter of clause 44, wherein the locking assembly fixes the therapeutic catheter relative to the support catheter prior to the one or more therapeutic elements extending from the delivery lumen to the deployed configuration.

Clause 46. The therapeutic catheter of clause 44 or 45, wherein the locking assembly fixes the therapeutic catheter relative to the support catheter prior to the one or more therapeutic elements extending from the delivery lumen.

Clause 47. The therapeutic catheter of any of clauses 44 to 46, wherein the locking assembly fixes the therapeutic catheter relative to the support catheter until the one or more therapeutic elements retract into the delivery lumen to a delivery configuration.

Clause 48. The therapeutic catheter of any of clauses 44 to 47, wherein the locking assembly fixes the therapeutic catheter relative to the support catheter until the one or more therapeutic elements retract into the delivery lumen.

Clause 49. The therapeutic catheter of any of clauses 44 to 48, wherein the locking assembly is configured to exert a radially inward force on an outer surface of an elongated body of the support catheter to fix the therapeutic catheter relative to the support catheter.

Clause 50. The therapeutic catheter of any of clauses 44 to 49, wherein the locking assembly is configured to exert a radially outward force on an inner surface of an elongated body of the support catheter to fix the therapeutic catheter relative to the support catheter.

Clause 51. The therapeutic catheter of any of clauses 44 to 50, wherein the locking assembly and the actuation assembly are configured to receive an actuation force and translate the actuation force into a locking force to fix the therapeutic catheter relative to the support catheter.

Clause 52. The therapeutic catheter of any of clauses 44 to 51, further comprising an actuation element configured to:
operate the locking assembly to fix the therapeutic catheter relative to the support catheter; and
operate the actuation assembly to deploy the one or more therapeutic elements.

Clause 53. The therapeutic catheter of clause 52, wherein the actuation element is configured to simultaneously:
operate the locking assembly to fix the therapeutic catheter relative to the support catheter; and
operate the actuation assembly to extend the one or more therapeutic elements from the delivery lumen into the deployed configuration.

Clause 54. The therapeutic catheter of clause 52, wherein the actuation element is configured to:
at a first position, operate the locking assembly to fix the therapeutic catheter relative to the support catheter; and
at a second position, operate the actuation assembly to extend the one or more therapeutic elements from the delivery lumen into the deployed configuration.

Clause 55. The therapeutic catheter of any of clauses 52 to 54, wherein the actuation element comprises a pressable button configured to move between a locked position and an unlocked position.

Clause 56. The therapeutic catheter of any of clauses 52 to 54, wherein the actuation element comprises a rotatable knob configured to rotate between a locked position and an unlocked position.

Clause 57. The therapeutic catheter of any of clauses 52 to 54, wherein the actuation element comprises a slidable sheath configured to move between a locked position and an unlocked position.

Clause 58. The therapeutic catheter of any of clauses 44 to 51, wherein the locking assembly is configured to permit operation of the actuation assembly.

Clause 59. The therapeutic catheter of clause 58, wherein the locking assembly further comprises a movable element configured to:
move between a locked position and an unlocked position;
when in the locked position, permit operation of the actuation assembly by exposing an actuation element configured to operate the actuation assembly, and
when in the unlocked position, permit movement of the therapeutic catheter relative to the support catheter.

Clause 60. The therapeutic catheter of any of clauses 44 to 59,
wherein each of the one or more therapeutic elements includes a needle, and
wherein, in a deployed configuration, each needle of the one or more therapeutic elements is configured to extend at least partially through a wall of the vasculature at the treatment site.

Clause 61. The therapeutic catheter of clause 60, wherein the one or more therapeutic elements are each configured to deliver a chemical agent to the treatment site.

Clause 62. The therapeutic catheter of clause 61, wherein the chemical agent is configured to modulate activity of at least one renal nerve in perivascular tissue.

Clause 63. The therapeutic catheter of any of clauses 60 to 62,
wherein each of the one or more therapeutic elements further comprises a guide tube, and
wherein the needle of each therapeutic element is configured to be moved relative to the corresponding guide tube.

Clause 64. The therapeutic catheter of any of clauses 44 to 63, wherein the therapeutic catheter is an ablation catheter.

Clause 65. The therapeutic catheter of any of clauses 44 to 64, wherein each of the one or more therapeutic elements comprises an electrode.

Clause 66. A medical assembly, comprising:
a support catheter comprising an elongated body defining a guide lumen and configured to position directly within vasculature of a patient;
a therapeutic catheter comprising:
an elongated body configured to move within the guide lumen;
one or more therapeutic elements at a distal portion of the elongated body, wherein the one or more therapeutic elements are configured to deliver therapy to a treatment site of the patient; and
an actuation assembly configured to operate the one or more therapeutic elements to deliver the therapy to the treatment site; and
a locking assembly coupled to at least one of the support catheter or the therapeutic catheter, wherein the locking assembly is configured to fix the therapeutic catheter relative to the support catheter while the one or more therapeutic elements deliver the therapy to the treatment site.

Clause 67. The medical assembly of clause 66,
wherein the locking assembly is coupled to the support catheter, and
wherein the locking assembly is configured to exert a radially inward force on an outer surface of the elongated body of the therapeutic catheter to fix the therapeutic catheter relative to the support catheter.

Clause 68. The medical assembly of clause 66,
wherein the locking assembly is coupled to the therapeutic catheter, and
wherein the locking assembly is configured to exert a radially outward force on an inner surface of the elongated body of the support catheter to fix the therapeutic catheter relative to the support catheter.

Clause 69. The medical assembly of any of clauses 66 to 68, wherein the locking assembly and the actuation assembly are configured to receive an actuation force and translate the actuation force into a locking force to fix the therapeutic catheter relative to the support catheter.

Clause 70. The medical assembly of any of clauses 66 to 69, further comprising an actuation element configured to:
operate the locking assembly to fix the therapeutic catheter relative to the support catheter; and
operate the actuation assembly to deliver the therapy from the one or more therapeutic elements.

Clause 71. The medical assembly of clause 70, wherein the actuation element is configured to:
at a first position, operate the locking assembly to fix the therapeutic catheter relative to the support catheter; and
at a second position, operate the actuation assembly to deliver the therapy from the one or more therapeutic elements.

Clause 72. The medical assembly of clause 70 or 71, wherein the actuation element comprises a pressable button configured to move between a locked position and an unlocked position.

Clause 73. The medical assembly of clause 70 or 71, wherein the actuation element comprises a rotatable knob configured to rotate between a locked position and an unlocked position.

Clause 74. The medical assembly of clause 70 or 71, wherein the actuation element comprises a slidable sheath configured to move between a locked position and an unlocked position.

Clause 75. The medical assembly of any of clauses 66 to 68, wherein the locking assembly is configured to permit operation of the actuation assembly.

Clause 76. The medical assembly of clause 75, wherein the locking assembly further comprises a movable element configured to:
move between a locked position and an unlocked position;
when in the locked position, permit operation of the actuation assembly by exposing an actuation element configured to operate the actuation assembly, and
when in the unlocked position, permit movement of the therapeutic catheter relative to the support catheter.

Clause 77. The medical assembly of any of clauses 66 to 76,
wherein each of the one or more therapeutic elements includes a needle, and
wherein, in a deployed configuration, each needle of the one or more therapeutic elements is configured to extend at least partially through a wall of the vasculature at the treatment site.

Clause 78. The medical assembly of any of clauses 66 to 77, wherein the therapeutic catheter is an ablation catheter.

Clause 79. The medical assembly of any of clauses 66 to 78, wherein each of the one or more therapeutic elements comprises an electrode.

Clause 80. A method comprising:
positioning a distal portion of an elongated body of a support catheter in vasculature of a patient, wherein the elongated body of the support catheter defines a guide lumen;
advancing a distal portion of an elongated body of a therapeutic catheter through the guide lumen to a treatment site of the patient, wherein the distal portion of the therapeutic catheter comprises one or more therapeutic elements configured to deliver therapy to a treatment site of the patient;
fixing the therapeutic catheter relative to the support catheter; and
delivering therapy from the one or more therapeutic elements.

Clause 81. The method of clause 80, wherein the locking assembly fixes the therapeutic catheter relative to the support catheter prior to delivering therapy from the one or more therapeutic elements.

Clause 82. The method of clause 80 or 81, wherein the locking assembly fixes the therapeutic catheter relative to the support catheter until the one or more therapeutic elements stop delivering therapy.

Clause 83. The method of any of clauses 80 to 82, further comprising:
operating an actuation assembly to deliver therapy from the one or more therapeutic elements; and
operating a locking assembly to fix the therapeutic catheter relative to the support catheter.

Clause 84. The method of clause 83,
wherein, prior to operation, the locking assembly is coupled to the support catheter, and
wherein operating the locking assembly comprises exerting a radially inward force on an outer surface of the elongated body of the therapeutic catheter to fix the therapeutic catheter relative to the support catheter.

Clause 85. The method of clause 83,
wherein, prior to operation, the locking assembly is coupled to the therapeutic catheter, and
wherein operating the locking assembly comprises exerting a radially outward force on an inner surface of the elongated body of the support catheter to fix the therapeutic catheter relative to the support catheter.

Clause 86. The method of any of clauses 83 to 85, further comprising operating an actuation element to operate the actuation assembly and the locking assembly.

Clause 87. The method of clause 86, wherein operating the actuation element comprises simultaneously:
operating the locking assembly to fix the therapeutic catheter relative to the support catheter; and
operating the actuation assembly to deliver therapy from the one or more therapeutic elements.

Clause 88. The method of clause 86, wherein operating the actuation element comprises:
at a first position, operating the locking assembly to fix the therapeutic catheter relative to the support catheter; and
at a second position, operating the actuation assembly to deliver therapy from the one or more therapeutic elements.

Clause 89. The method of any of clauses 86 to 88, wherein the actuation element comprises a pressable button.

Clause 90. The method of any of clauses 86 to 88, wherein the actuation element comprises a rotatable knob.

Clause 91. The method of any of clauses 86 to 88, wherein the actuation element comprises a slidable sheath.

Clause 92. The method of any of any of clauses 83 to 85, wherein operating the locking assembly permits operation of the actuation assembly.

Clause 93. The method of clause 92, wherein operating the locking assembly further comprises:
moving a movable element from an unlocked position to a locked position to fix the therapeutic catheter relative to the support catheter and expose an actuation element; and
operating the actuation element to operate the actuation assembly.

Clause 94. The method of any of clauses 80 to 93,
wherein the one or more therapeutic elements comprise one or more electrodes, and
wherein delivering the therapy comprises delivering energy from the one or more electrodes.

Clause 95. The method of any of clauses 80 to 93,
wherein the one or more therapeutic elements comprise one or more apertures fluidically coupled to a pressurized fluid source, and
wherein delivering the therapy further comprises delivering a chemical agent from the one or more apertures.

Clause 96. A therapeutic catheter comprising:
an elongated body configured to move within a guide lumen of a support catheter;
one or more therapeutic elements at a distal portion of the elongated body,
wherein the one or more therapeutic elements are configured to deliver therapy to a treatment site of a patient;
an actuation assembly configured to deliver the therapy from the one or more therapeutic elements; and
a locking assembly configured to fix the therapeutic catheter relative to the support catheter while the one or more therapeutic elements deliver therapy to the treatment site.

Clause 97. The therapeutic catheter of clause 96, wherein the locking assembly fixes the therapeutic catheter relative to the support catheter prior to delivering therapy from the one or more therapeutic elements.

Clause 98. The therapeutic catheter of clause 96 or 97, wherein the locking assembly fixes the therapeutic catheter relative to the support catheter until the one or more therapeutic elements stop delivering therapy.

Clause 99. The therapeutic catheter of any of clauses 96 to 98, wherein the locking assembly is configured to exert a radially inward force on an outer surface of an elongated body of the support catheter to fix the therapeutic catheter relative to the support catheter.

Clause 100. The therapeutic catheter of any of clauses 96 to 98, wherein the locking assembly is configured to exert a radially outward force on an inner surface of an elongated body of the support catheter to fix the therapeutic catheter relative to the support catheter.

Clause 101. The therapeutic catheter of any of clauses 96 to 100, wherein the locking assembly and the actuation assembly are configured to receive an actuation force and translate the actuation force into a locking force to fix the therapeutic catheter relative to the support catheter.

Clause 102. The therapeutic catheter of any of clauses 96 to 101, further comprising an actuation element configured to:
operate the locking assembly to fix the therapeutic catheter relative to the support catheter; and
operate the actuation assembly to deliver therapy from the one or more therapeutic elements.

Clause 103. The therapeutic catheter of clause 101, wherein the actuation element is configured to simultaneously:
operate the locking assembly to fix the therapeutic catheter relative to the support catheter; and
operate the actuation assembly to deliver therapy from the one or more therapeutic elements.

Clause 104. The therapeutic catheter of clause 101, wherein the actuation element is configured to:
at a first position, operate the locking assembly to fix the therapeutic catheter relative to the support catheter; and
at a second position, operate the actuation assembly to deliver therapy from the one or more therapeutic elements from the delivery lumen into the deployed configuration.

Clause 105. The therapeutic catheter of any of clauses 102 to 104, wherein the actuation element comprises a pressable button configured to move between a locked position and an unlocked position.

Clause 106. The therapeutic catheter of any of clauses 102 to 104, wherein the actuation element comprises a rotatable knob configured to rotate between a locked position and an unlocked position.

Clause 107. The therapeutic catheter of any of clauses 102 to 104, wherein the actuation element comprises a slidable sheath configured to move between a locked position and an unlocked position.

Clause 108. The therapeutic catheter of any of clauses 96 to 101, wherein the locking assembly is configured to permit operation of the actuation assembly.

Clause 109. The therapeutic catheter of clause 108, wherein the locking assembly further comprises a movable element configured to:
move between a locked position and an unlocked position;
when in the locked position, permit operation of the actuation assembly by exposing an actuation element configured to operate the actuation assembly, and
when in the unlocked position, permit movement of the therapeutic catheter relative to the support catheter.

Clause 110. The therapeutic catheter of any of clauses 96 to 109, wherein each of the one or more therapeutic elements comprises an electrode.

Clause 111. The therapeutic catheter of any of clause 96 to 109, wherein each of the one or more therapeutic elements comprises an aperture configured to deliver a chemical agent to the treatment site of the patient.

Clause 112. A locking assembly, comprising:
a housing configured to couple to at least one of a support catheter or a therapeutic catheter; and
a moveable element configured to move relative to the housing between a locked position and an unlocked position and, in the locked position, fix the therapeutic catheter relative to the support catheter while one or more therapeutic elements of the therapeutic catheter are at least one of: extending from a delivery lumen of the therapeutic catheter, or delivering therapy to a tissue.

Clause 113. The locking assembly of clause 112,
wherein the housing is configured to couple to the support catheter, and
wherein the moveable element is configured to exert a radially inward force on an outer surface of an elongated body of the therapeutic catheter to fix the therapeutic catheter relative to the support catheter.

Clause 114. The locking assembly of clause 112,
wherein the housing is configured to couple to the therapeutic catheter, and
wherein the moveable element is configured to exert a radially outward force on an inner surface of an elongated body of the support catheter to fix the therapeutic catheter relative to the support catheter.

Clause 115. The locking assembly of any of clauses 112 to 114, wherein the locking assembly is configured to translate an actuation force on an actuation assembly of the therapeutic catheter into a locking force to fix the therapeutic catheter relative to the support catheter.

Clause 116. The locking assembly of clause 115, wherein the moveable element is configured to:
interface with an actuation element of the actuation assembly; and
in response to receiving the actuation force received by the actuation element, fix the therapeutic catheter relative to the support catheter.

Clause 117. The locking assembly of clause 115, wherein the moveable element is configured to permit operation of the actuation assembly.

Clause 118. The locking assembly of clause 117, wherein the moveable element is further configured to:
move between a locked position and an unlocked position;
when in the locked position, permit operation of the actuation assembly by exposing the actuation element configured to operate the actuation assembly, and
when in the unlocked position, permit movement of the therapeutic catheter relative to the support catheter.

1. A medical assembly, comprising:
a support catheter comprising an elongated body defining a guide lumen and configured to position directly within vasculature of a patient;
a therapeutic catheter comprising:
an elongated body defining a delivery lumen and configured to move within the guide lumen;
one or more therapeutic elements at a distal portion of the elongated body of the therapeutic catheter, wherein the one or more therapeutic elements are configured to extend from the delivery lumen into a deployed configuration at a treatment site of the patient; and
an actuation assembly configured to deploy the one or more therapeutic elements to extend from the delivery lumen into the deployed configuration; and
a locking assembly coupled to at least one of the support catheter or the therapeutic catheter, wherein the locking assembly is configured to fix the therapeutic catheter relative to the support catheter while the one or more therapeutic elements are extended from the delivery lumen.

2. The medical assembly of claim 1, wherein the locking assembly is configured to fix the therapeutic catheter relative to the support catheter prior to the one or more therapeutic elements extending from the delivery lumen to the deployed configuration.

3. The medical assembly of claim 1, wherein the locking assembly is configured to fix the therapeutic catheter relative to the support catheter prior to the one or more therapeutic elements extending from the delivery lumen.

4. The medical assembly of claim 1, wherein the locking assembly is configured to fix the therapeutic catheter relative to the support catheter until the one or more therapeutic elements retract into the delivery lumen to a delivery configuration.

5. The medical assembly of claim 1, wherein the locking assembly is configured to fix the therapeutic catheter relative to the support catheter until the one or more therapeutic elements retract into the delivery lumen.

6. The medical assembly of claim 1,
wherein the locking assembly is coupled to the support catheter, and
wherein the locking assembly is configured to exert a radially inward force on an outer surface of the elongated body of the therapeutic catheter to fix the therapeutic catheter relative to the support catheter.

7. The medical assembly of claim 1,
wherein the locking assembly is coupled to the therapeutic catheter, and
wherein the locking assembly is configured to exert a radially outward force on an inner surface of the elongated body of the support catheter to fix the therapeutic catheter relative to the support catheter.

8. The medical assembly of claim 1, wherein the locking assembly and the actuation assembly are configured to receive an actuation force and translate the actuation force into a locking force to fix the therapeutic catheter relative to the support catheter.

9. The medical assembly of claim 1, further comprising an actuation element configured to:
operate the locking assembly to fix the therapeutic catheter relative to the support catheter; and
operate the actuation assembly to deploy the one or more therapeutic elements.

10. A method comprising:
positioning a distal portion of an elongated body of a support catheter in vasculature of a patient, wherein the elongated body of the support catheter defines a guide lumen;
advancing a distal portion of an elongated body of a therapeutic catheter through the guide lumen to a treatment site of the patient, wherein the elongated body of the therapeutic catheter defines a delivery lumen, and wherein the distal portion of the therapeutic catheter comprises one or more therapeutic elements configured to extend from the delivery lumen into a deployed configuration at the treatment site;
fixing the therapeutic catheter relative to the support catheter; and
deploying the one or more therapeutic elements to extend from the delivery lumen into the deployed configuration.

11. A therapeutic catheter comprising:
an elongated body defining a delivery lumen and configured to move within a guide lumen of a support catheter;
one or more therapeutic elements at a distal portion of the elongated body, wherein the one or more therapeutic elements are configured to extend from the delivery lumen into a deployed configuration at a treatment site of the patient;
an actuation assembly configured to deploy the one or more therapeutic elements to extend from the delivery lumen into the deployed configuration; and
a locking assembly configured to fix the therapeutic catheter relative to the support catheter while the one or more therapeutic elements are extended from the delivery lumen.

12. The therapeutic catheter of claim 11, wherein the locking assembly is configured to fix the therapeutic catheter relative to the support catheter prior to the one or more therapeutic elements extending from the delivery lumen to the deployed configuration.

13. The therapeutic catheter of claim 11, wherein the locking assembly is configured to fix the therapeutic catheter relative to the support catheter prior to the one or more therapeutic elements extending from the delivery lumen.

14-20. (canceled)

21. The medical assembly of claim 1, wherein the one or more therapeutic elements includes a plurality of needles, and wherein, in the deployed configuration, at least one needle of the plurality of needles is configured to extend at least partially through a wall of the vasculature at the treatment site.

22. The medical assembly of clause 21, wherein the plurality of needles is configured to deliver a chemical agent to the treatment site, wherein the chemical agent is configured to modulate activity of at least one nerve in perivascular tissue.

23. The medical assembly of claim 21, wherein the one or more therapeutic elements further comprises a plurality of guide tubes, and wherein each needle of the plurality of needles is configured to be moved relative to a corresponding guide tube of the plurality of guide tubes.

24. The method of claim 10, wherein fixing the therapeutic catheter relative to the support catheter comprises fixing the therapeutic catheter relative to the support catheter prior to deploying the one or more therapeutic elements.

25. The method of claim 10, wherein fixing the therapeutic catheter relative to the support catheter comprises fixing the therapeutic catheter relative to the support catheter with a locking assembly coupled to the support catheter, wherein the locking assembly is configured to exert a radially inward force on an outer surface of the elongated body of the therapeutic catheter to fix the therapeutic catheter relative to the support catheter.

26. The method of claim 10, wherein fixing the therapeutic catheter relative to the support catheter comprises fixing the therapeutic catheter relative to the support catheter with a locking assembly coupled to the support catheter, wherein the locking assembly is configured to exert a radially outward force on an outer surface of the elongated body of the therapeutic catheter to fix the therapeutic catheter relative to the support catheter.

27. The therapeutic catheter of claim 11, wherein the one or more therapeutic elements includes a plurality of needles, and wherein, in the deployed configuration, at least one needle the plurality of needles is configured to extend at least partially through a wall of the vasculature at the treatment site.
